(19) United States
(12) Patent Application Publication
(10) Pub. No.: US 2021/0371828 A1
(43) Pub. Date: Dec. 2, 2021

(54) INDUCED PLURIPOTENT CELL COMPRISING A CONTROLLABLE TRANSGENE FOR CONDITIONAL IMMORTALIZATION

(71) Applicant: ReNeuron Limited, Pencoed, Bridgend, Wales (GB)

(72) Inventors: Steve PELLS, Pencoed (GB); Randolph CORTELING, Pencoed (GB); John Sinden, Pencoed (GB)

(21) Appl. No.: 17/284,428

(22) PCT Filed: Oct. 11, 2019
(86) PCT No.: PCT/GB2019/052908

§ 371 Date: Apr. 9, 2021

(30) Foreign Application Priority Data
Oct. 12, 2018 (GB) 1816670.2
Oct. 17, 2018 (GB) 1816934.2

(51) Int. Cl. C12N 5/074; A61K 35/545

(52) U.S. Cl. CPC C12N 5/0696; A61K 35/545; C12N 2510/04; C12N 2506/45; C12N 2501/608; C12N 2506/08; C12N 2501/603; C12N 2501/602; C12N 2501/604; C12N 2501/606

(57) ABSTRACT

The invention relates to induced pluripotent stem cells that are generated from cells, for example Adult Stem Cells, that are conditionally-immortalisable. In particular, the invention relates to induced pluripotent stem cells generated from stem cell lines comprising a controllable transgene for conditional immortalisation, and the progeny of those induced pluripotent stem cells. Induced pluripotent stem cells, progeny cells derived from those pluripotent cells, compositions comprising those cells, methods of making all of those cells, and uses of all of those cells are also described.

### FIELD OF THE INVENTION

This invention relates to induced pluripotent stem cells that are generated from cells, for example adult stem cells, that are conditionally-immortalisable. In particular, the invention relates to induced pluripotent stem cells generated from cells comprising a controllable transgene for conditional immortalisation, and the progeny of those induced pluripotent stem cells.

### BACKGROUND OF THE INVENTION

Human Pluripotent Stem Cells (hPSCs) are defined by the property of pluripotency: the property of being capable of differentiation into any cell type found in the body. They include embryonic stem cells (hESCs), derived from the inner cell mass of the early-stage embryo (blastocyst, approximately day 6.5 post-fertilisation), and induced pluripotent cells (hiPSCs), generated by reprogramming of somatic cells to a pluripotent phenotype by the transduction and exogenous expression of certain transcription factors.

The canonical set of such transcription factors capable of reprogramming to pluripotency is known as OKSM (OCT4, KLF4, SOX2, C-MYC), but other factors are known which may substitute for 0, K, S or M, or modulate the efficiency with which reprogramming, a stochastic process, occurs.

Typically, low-passage primary cells are the preferred substrate for reprogramming to generate induced pluripotent stem cells (iPSCs). Such cells have the advantages that they tend to divide faster than higher passage cells; non-dividing cells are refractory to reprogramming. They are also more likely to be euploid. Adult Stem Cells (ASCs) also provide a promising substrate for reprogramming to pluripotency, often requiring fewer transcription factors and a more modest reprogramming event, due to endogenous expression of reprogramming factors (e.g. SOX2, KLF4) and a more open chromatin structure associated with (pluri-/multi-) potency.

There are some examples (mostly EBV-immortalised blood cells) of iPSCs being generated from immortal mammalian cells rather than primary cells. Since such cells are typically immortalised by the stable genomic integration of EBV or an oncogene such as the simian virus 40 large T antigen, their clinical utility is doubtful. The 293FT cell line, for example, stably expressed the SV40 large T antigen and whilst phenotype changes upon transfection of reprogramming transcription factors, it generates anomalous colonies rather than true iPSCs. iPSC lines derived from such immortalised cells are thus typically limited to in vitro applications such as disease modelling, drug discovery and developmental studies.

Furthermore, a study by Skvortsova et al (Oncotarget, 2018, Vol. 9 (No. 81), pp 35241-35250) reports that immortalised murine fibroblast cell lines are refractory to reprogramming to a pluripotent state, and that aneuploidies associated with immortalisation and in vitro selection are unlikely to be the cause of such refractoriness. There are therefore significant technical challenges when seeking to identify cells suitable for reprogramming to a pluripotent state, and at least some immortalised cells are refractory to reprogramming.

Pluripotent stem cells are stable and may be cultured indefinitely in vitro. However, there are challenges to their clinical application such as their capability of teratoma formation and the issue that most differentiation protocols result in less than 100% differentiation to the desired endpoint. There thus remains the formal risk of teratoma formation from residual pluripotent cells in the therapeutic population, and the issue of co-transfer of undesired cell types to the patient. Such undesired subpopulations may have either neutral or negative effects, even if only a reduction in the efficiency of the therapy. This is exacerbated by the fact that the desired therapeutic cell type is often not the terminally-differentiated cell type whose chronic or acute loss causes pathology in the patient, but rather a late tissue progenitor/adult stem cell population which gives rise to the final cell type in the appropriate tissues of the patient. Late progenitor populations are often not stable in in vitro culture, and thus in addition to the purity issues noted above their scalable production at acceptable levels of purity for clinical use is a non-trivial challenge, even if in theory their production from pluripotent cells is effectively unlimited.

Such progenitor cell populations are also typically very difficult to handle. If they are isolated from either the patient or another person prior to transplant (e.g., bone marrow cells), difficulties associated with the very limited availability of material, the requirement for both persons to be available for operations at the same time and place, availability of a suitable (e.g. immunocompatible) donor, lack of transferred material purity and QC all serve to limit the generalisability of such treatments. Theoretically, the possibility of generating such cell populations by differentiating hPSCs such as hiPSCs would ameliorate such issues, but they present other challenges. Significantly, progenitor populations are difficult to handle in vitro and are not stable over time. As such, their homogenous, GMP-compliant scalable production is typically not possible even if GMP (medical) quality iPSCs are available. Most differentiation protocols are not 100% efficient, so that contaminating subpopulations are likely to be present in the therapeutic cell population.

There remains a need to generate stem cells with clinical utility.

### SUMMARY OF THE INVENTION

The present invention is based on the surprising realisation that induced Pluripotent Stem Cell (iPSC) technology can be improved by generating iPSCs from conditionally-immortalised cells, in particular conditionally-immortalised stem cells.

A first aspect of the invention provides an induced pluripotent stem cell comprising a controllable transgene for conditional immortalisation. The controllable transgene will typically be able to conditionally-immortalise downstream (more differentiated) cells that are derived from the induced pluripotent stem cell. These downstream cells may otherwise be difficult to handle, so the presence of the conditionally-immortalising transgene is an improvement.

A second aspect of the invention provides a pluripotent stem cell that is obtainable or obtained from a conditionally-immortalised cell, typically a conditionally-immortalised stem cell.

A third aspect of the invention provides a method of producing a pluripotent stem cell, comprising the step of reprogramming a conditionally-immortalised cell, typically a conditionally-immortalised stem cell. The method may further comprise subsequent steps to generate different cell types from the pluripotent stem cell.

Further aspects of the invention relate to cells produced by any method of the invention, and microparticles produced by any of those cells.

The inventors have found that reprogramming conditionally-immortalised cells such as stem cells, for example cells from the CTX0E03 or STR0C05 cell line, to pluripotency will allow the generation of other adult stem cell or tissue progenitor populations. Once the pluripotent cell is generated, conventional differentiation protocols can be used to provide cells of any desired lineage, for example the ectoderm, endoderm or mesoderm lineage. In certain embodiments, the pluripotent cells are directed down a lineage that is different from the lineage of the original conditionally-immortalised stem cell. In certain embodiments, the induced pluripotent cell can be differentiated into a mesenchymal stem cell, a neural stem cell, or a haematopoietic stem cell, including further differentiation to cells of the immune system such as T lymphocytes, NK cells and dendritic cells. In certain embodiments, the induced pluripotent cells may be differentiated into a somatic (adult) stem cell, a multipotent cell, an oligopotent cell or a unipotent cell; or a terminally-differentiated cell.

The Examples below demonstrate that iPSCs, generated according to the invention from different conditionally-immortalised cells, are pluripotent and are able to enter the endoderm, mesoderm and ectoderm lineages. The Examples further show that adult stem cells (MSCs) can be generated from the iPSCs of the invention. These MSCs are shown to be multipotent, and able to differentiate into cartilage, fat and bone cells.

Differentiation of the induced pluripotent cells of the invention into functional cells of the immune system, is also described. These immune cells include T cells, B cells, Natural Killer (NK) cells and Dendritic cells. As will be understood by the skilled person, these cells will usually be arrived at via the haematopoietic lineage.

A cell, e.g. a stem cell, can be rendered conditionally-immortalisable through the c-myc-ER^{TAM} transgene. This transgene has already been shown to permit the stable, scalable production of stem cell lines, such as the neural stem cell lines CTX0E03 and STR0C05, by the addition of 4-hydroxytamoxifen to the cell culture medium, which promotes growth and cell division without any change in phenotype.

The conditionally-immortalised stem cell is typically an adult stem cell, also referred to as a somatic stem cell. For example, it could be a neural stem cell, such as a cell from the CTX0E03 stem cell line. The CTX0E03 neural stem cell line has been deposited by the applicant (ReNeuron Limited) at the European Collection of Authenticated Cell Cultures (ECACC), Porton Down, UK and having ECACC Accession No. 04091601. In other embodiments, the neural stem cell line may be the “STR0005” cell line, the “HPCOA07” cell line (also deposited by the applicant at ECACC) or the neural stem cell line disclosed in Miljan et al Stem Cells Dev. 2009.

The conditionally-immortalised stem cell is reprogrammed to pluripotency. Inducing the pluripotent phenotype typically involves introducing products of specific sets of pluripotency-associated genes, or “reprogramming factors”, into a given cell type. The original set of reprogramming factors (also dubbed Yamanaka factors) are the transcription factors Oct4, Sox2, cMyc, and Klf4. The reprogramming factors are typically introduced into the cell using viral or episomal vectors, as is well-known in the art.

Viral vectors suitable for introducing reprogramming factors into a cell include lentivirus, retrovirus and Sendai-virus. Other techniques for introducing reprogramming factors include mRNA transfection.

It has been surprisingly observed that only one transcription factor is required to reprogram certain conditionally-immortalised stem cells, such as CTX0E03, to pluripotency. For example, FIGS. 2B-D show that OCT4 alone can induce pluripotency of CTX0E03. Combinations of transcription factors that were observed to achieve pluripotency include: OCT4 and SOX2; OCT, KLF4 and SOX2; OCT4, KLF4, SOX2 and MYC. Accordingly, reprogramming factors that comprise or consist of these combinations are provided for use in the present invention. Each of the combinations of factors that successfully induce pluripotency in FIG. 2C is provided as a separate embodiment of the invention. Reprogramming factors for use in inducing conditionally-immortalised stem cells to pluripotency may comprise or consist of an exemplified combination.

Other immortalisation factors are known in the art and will be apparent to the skilled person. Any suitable combination of factors can be used, which is well within the ability of one skilled in the art. For example, reprogramming with small molecule inhibitors is known in the art, while NANOG and TET1 are known as other suitable transcription factors. By way of example, Thomson and colleagues used NANOG, KLF4, SOX 2 and LIN28 as an alternative to OKSM. In another example, TET1 has been shown to be capable of substituting for OCT4.

As the activity of the c-myc-ER^{TAM} transgene recapitulates cellular MYC activity, vectors expressing the MYC oncogene are dispensable when reprogramming c-myc-ER^{TAM} inducibly-immortalised stem cells, because if required this activity can be provided by the provision of 4-OHT to the medium to activate the c-myc-ER^{TAM} fusion protein. Accordingly, in some embodiments, MYC (for example a MYC reprogramming vector) is not used as a separate reprogramming factor. The skilled person will of course be aware that conditional immortalisation systems using genes other than MYC, may require exogenous MYC to reprogram them.

The induced pluripotent cells can be differentiated into any desired cell type. Techniques for determining a cell lineage or cell type are well known in the art. Typically, these techniques involve the determination of markers of differentiation on either the cell surface (and/or the absence of markers of pluripotency such as Oct4) or internally, such as the presence of lineage-specific transcription factors, cell morphology and function. For example, pluripotent stem cells typically are positive for the canonical pluripotent transcription factor OCT4, and the cell surface antigens TRA-1-60 and SSEA-4, but do not express the early differentiation marker SSEA-1. Markers of the endoderm lineage include GATA6, AFP or HNF-alpha. Other endoderm markers can include one or more of Claudin-6, Cytokeratin 19, EOMES, SOX7 and SOX17. Markers of the mesoderm lineage include BMP2, Brachyury or VEGF. Other mesoderm markers can include one or more of Activin A, GDF-1, GDF-3, and TGF-beta. Markers of the ectoderm lineage include PAX6, Nestin or TubIII. Other ectoderm markers can include one or more of Noggin, PAX2 and chordin.

Differentiation into different lineages is shown for example in FIG. 3D. Differentiation of CTX-iPSCs and STROC-iPSCs into endoderm, mesoderm and ectoderm lineages is also demonstrated in Example 2 (FIG. 7) and Example 3 (FIG. 9). Example 2 uses the following markers:

| Lineage | Marker |
|---|---|
| Endoderm | SOX17 |
| | FOXA2 |
| Mesoderm | BRACHYURY |
| | CXCR4 |
| Ectoderm | PAX6 |
| | NESTIN |

The pluripotent cells can be differentiated into any desired cell type. This can include a mesenchymal stem cell, a neural stem cell, or a haematopoietic stem cell. In another embodiment, a somatic (adult) stem cell results from differentiation of the induced pluripotent cell of the invention. In other embodiments, the cell that is provided by the method is a multipotent cell, an oligopotent cell or a unipotent cell. An example of this embodiment would be the production of progenitor cells, for example neuronal progenitor cells. Neuronal progenitor cells have been described as being potentially of use in treatment of neurodegenerative diseases, by Nistor and colleagues in PloS One (2011) vol. 6 e20692. The cell that results can also be fully differentiated, using known techniques for differentiation, into a terminally-differentiated cell. An example of this embodiment is the differentiation to and scalable production of medium spiny neurons, as are lost in Huntington's disease, as described by Carri and colleagues (2013); see Stem Cell Review and Reports, DOI 10.1007/s12015-013-9441-8. In a particular embodiment, the haematopoietic stem cell can be differentiated into a T cell, an NK cell and/or a dendritic cell. T cells are therefore provided as one embodiment. Natural Killer cells are provided as another embodiment. Dendritic cells are further provided.

Differentiation of CTX-iPSCs into mesenchymal stem cells is demonstrated in Example 1 and FIG. 5. In this example the MSC phenotype is identified by the presence of the markers CD73, CD90 and CD105, but not CD14, CD20, CD34 or CD45.

Differentiation of the CTX-iPSC-MSCs into cartilage, fat and bone cells is demonstrated in Example 3 and FIG. 10.

Reactivation (if necessary) of the c-myc-ER^{TAM} transgene followed by addition of 4-OHT to the medium should permit indefinite growth of the derived cell population. By analogy with CTX0E03 itself, this is expected to allow the scalable production of effectively unlimited quantities of a therapeutically useful cell population in vitro which may be used as an “off-the-shelf” therapy for any condition characterised by acute or chronic cell loss for which cell therapies either do not exist, or are limited by tissue donor availability or technical limitations of differentiation protocols from hPSCs.

The methods of the invention can involve further processing, culture or formulation steps that may be necessary to provide the desired product. In certain embodiments, a method of the invention can include one or more of the following steps, typically at the end of the method:
culturing the cells that result from the method;
passaging the cells that result from the method;
harvesting or collecting the cells that result from the method;
packaging the cells that result from the method into one or more containers; and/or
formulating the cells that result from the method with one or more excipients,
stabilisers or preservatives.

The conditionally-immortalised stem cells, the pluripotent cells that result from reprogramming, and the more differentiated cells that can be obtained from the pluripotent cells, are typically isolated or purified. The microparticles (extracellular vesicles) produced by any of these cells, for example exosomes, are also typically isolated or purified.

The cells that are provided following differentiation, and the microparticles produced by them, can be used in therapy. Therapy will typically be of a disease or disorder in an individual in need thereof. The patient will typically be human.

In a further aspect, the invention provides a composition comprising: conditionally-immortalised stem cells; the pluripotent cells that result from reprogramming; the more differentiated cells that can be obtained from the pluripotent cells; or the microparticles produced by any of these cells; and a pharmaceutically acceptable excipient, carrier or diluent.

### BRIEF DESCRIPTION OF THE DRAWINGS

FIG. 1: Reprogramming of CTX cells to a pluripotent phenotype. (A) Schematic of CTX reprogramming process (HPSC medium: E8/Stemflex; hPSC substrate: LN-521/vitronectin-XF) and episomal plasmids driving OKSML expression which may be used for reprogramming (Epi5 kit, Invitrogen). CTX culture medium may be supplemented with 4-OHT or not, to provide MYC activity through the c-myc-ER^{TAM} transgene, as desired. Transfection may be achieved in a variety of ways, such as by lipofection, nucleofection or electroporation. (B) EGFP signal indicates transfection efficiency at 24 hours post-transfection. (C) Example young colonies of reprogrammed CTX cells with hPSC phenotype at day 15 post-transfection, showing very different cell and colony morphology from the parental CTX cells surrounding the colony. (D) Example 6-well plate showing hPSC-phenotypic (alkaline phosphatase-positive, red stained) colonies at day 21 endpoint.

FIG. 2 shows that CTX0E03 cells are reprogrammable with fewer factors. (A) Vectors expressing single factors, pCE-OCT3/4, pCE-SOX2 and pCE-KLF4; 4-OHT provision mimics MYC via c-myc-ER^{TAM}. (B) Inset: example AP-stained plate for colony counting. Main image: colony reprogrammed with transcription factor OCT4 alone. (C) Colony numbers obtained with different factor combinations (S-K: pCE-SK, M-L: pCE-UL, S: pCE-50X2, K: pCE-KLF4, M: 4-OHT→d 14). (D) Venn diagram showing combination effects (numbers: x colonies obtained; zeroes: no colonies).

FIG. 3 shows the pluripotent phenotype of CTX-iPSCs:
(A) Cell and colony morphology of CTX-iPSCs, wherein (ii, iii) are two examples of induced pluripotent CTX cell lines that recapitulate the dense colonies of small, closely-packed cells with prominent nucleoli characteristic of hPSCs, differing markedly from the neuronal phenotype of the parental CTX cells (i);
(B) CTX-iPSC lines express the enzymatic marker alkaline phosphatase (pink stain). Alkaline-phosphatase staining of established CTX-derived hIPSC lines, wherein Pink coloration indicates the cells are positive for the pluripotency marker TNAP and are thus capable of performing the enzyme-catalysed colour change reaction in vitro;
(C) Flow cytometry shows that CTX-iPSC lines express pluripotency-associated markers including the transcription factor OCT4, and the cell surface antigens SSEA-4 and TRA-1-60, but do not express the early differentiation marker SSEA-1. (i) Summary table for several CTX-iPSC lines, (ii) example data for one CTX-iPSC line: upper row, left to right: SSEA-1, OCT4, SSEA-4; lower row, left to right: TRA-1-60, forward v. side scatter, SSEA-4;
(D) RT-qPCR showing upregulation of lineage-specific markers upon in vitro differentiation to endoderm, mesoderm and ectoderm (individual CTX-iPSC lines indicated by colour).

FIG. 4 assesses the transgene locus in CTX-iPSCs. (A) Giemsa staining of parental CTX0E03 cells (top, 4 days, 2nd row, 10 days) and five CTX-iPSC lines at 4 days in G418 (3rd-7th rows) indicates expression activity of the c-myc-ER^{TAM}-associated NeoR gene. (B) Bisulphite-conversion of the CMV-IE promoter driving the c-myc-ER^{TAM} transgene shows the cytosine methylation state at the locus (white circle, unmethylated CpG; black circle, methylated CpG; comma, indeterminate read).

FIG. 5 shows the production of an exemplary therapeutic cell population derived from CTX-iPSCs. (A) Pluripotent CTX-iPSCs on Laminin-521 in mTeSR1 medium (standard culture conditions for preserving pluripotency in vitro). (B) Plastic-adherent candidate mesenchymal stem cells (MSCs) derived from cells in (A) in MSC medium (α-MEM, 10% FCS, 25 mM HEPES). (C) Flow cytometry of the CTX-iPSC-MSCs shows they express the MSC markers CD73, CD90 and CD105, but not CD14, CD20, CD34 or CD45, in accordance with ISCT criteria (blue, staining; red, isotype controls).

FIG. 6: Cellular reprogramming of CTX to pluripotency results in dramatic genome-wide changes in gene expression, shown here by examples of expression modulation in genes with significant roles in pluripotency and neural development. Single cell RNA sequence (transcriptome) data are shown for (see key, top left hand panel) three samples of CTX (green), three CTX-iPSC cell lines (blue) and the same CTX-iPSC lines having undergone differentiation along a cortical lineage (red). In the latter case, differentiation was halted at a point most closely recapitulating CTX itself, as defined by RT-qPCR analysis of a select set of neuroectodermal gene expression. Each panel is a “tSNE plot” of single cell gene expression data, with each dot in a “cloud” representing a single cell. Grey: no expression, orange: moderate expression; red: high expression. The plots show that pluripotency genes inactive in CTX have been activated in the reprogrammed cells: POU5F1, NANOG, UTF1, TET1, DPP4, TDGF1, ZSCAN10 and GAL. Importantly, of these genes only POU5F1 was provided exogenously during reprogramming, unequivocally confirming activation of the endogenous gene upon reprogramming. Conversely, several neural genes expressed by CTX are downregulated upon reprogramming to pluripotency (NOGGIN, ADAM12, NTRK3, PAX6), as is OCIAD2, a gene strongly expressed by CTX cells. Some genes important in neuroectodermal development, such as GLI3 and PAX6, are upregulated upon cortical differentiation of the pluripotent cells as they select a neuroectodermal fate.

FIG. 7 provides additional confirmation that CTX-iPSCs are pluripotent. Immunostaining for protein markers such as transcription factors identifying the three primary germ cell lineages, endoderm, mesoderm and ectoderm.

FIG. 8 shows the reprogramming of another conditionally immortalised adult stem cell type. The Figure shows successful reprogramming of another conditionally-immortalised adult stem cell (ASC) line, STR0005, derived from fetal striatal cells. Panels: A, a colony of reprogrammed STR0005 cells 24 days post-transfection with reprogramming factors; B, alkaline phosphatase (red)-stained STR0005 cells early in reprogramming, showing some cells beginning to express the pluripotency marker alkaline phosphatase; C, an established STR0005-iPSC line; D, AP-positive colonies appear at different frequencies in wells subjected to different transfection conditions; well 1 with no positive colonies was transfected with a GFP (non-reprogramming) plasmid as a control and had no reprogrammed cells, whereas wells 4 and 6 had few surviving cells; E, an established STR0C05-iPSC line is alkaline phosphatase positive, and F, is also positive for the pluripotency marker SSEA4 but negative for the early differentiation marker SSEA1.

FIG. 9: Confirmation of pluripotency of the STR0005-iPSCs. Differentiation to endoderm, mesoderm and ectoderm, shown by immunostaining confirming coexpression of protein markers (mostly transcription factors) identifying the three primary germ cell lineages.

FIG. 10: Confirmation of multipotency of adult stem cells derived from CTX-iPSCs. Candidate CTX-iPSC-derived mesenchymal stem cells (CTX-iPSC-MSCs) are multipotent. In addition to expression of a defined panel of cell surface marker proteins and adherence to tissue culture plastic (see main body of the Application), this Figure confirms their capacity to differentiate into cartilage (shown by alcian blue staining of glycosaminoglycans), fat (shown by staining of intracellular lipid droplets with oil red 0) and bone (shown by alizarin red staining of deposited calcium).

FIG. 11: Function of the conditional immortalisation transgene in adult stem cells differentiated from iPSCs created in turn by reprogramming of conditionally-immortalised cells. Flow cytometric profiles of CTX-iPSC-MSCs cultured to high passage (20 passages) in the presence or absence of 4-hydroxytamoxifen (4-OHT). This cell line is one that DNA methylation data suggest has a demethylated C-MYC-ER^{TAM} promoter, in turn implying that the promoter is active. This cell line appears to better maintain its cell surface marker profile when cell cycling is induced through the 4-OHT/C-MYC-ER^{TAM} system. CD90 and CD105 expression are more uniform and more highly expressed, and the negative markers CD14, 20, 34 and 45 are more consistently low. In the second panel, the 4-OHT-treated cells appear to be more efficient at generating bone upon differentiation, suggesting that 4-OHT/C-MYC-ER^{TAM}-driven cell cycling somewhat inhibits differentiation and associated loss of potency that might otherwise occur upon exit from the cell cycle.

FIG. 12: An example of CTX-iPSC-MSC lines cultured in the absence or presence of 4-OHT, showing improved and more consistent growth behaviour long-term when the C-MYC-ERTAM transgene is active (presence of 4-OHT).

FIG. 13: A second example of CTX-iPSC-MSC lines cultured in the absence or presence of 4-OHT, showing improved and more consistent growth behaviour long-term when the C-MYC-ER^{TAM} transgene is active (presence of 4-OHT).

### DETAILED DESCRIPTION OF THE INVENTION

The present inventors have surprisingly identified that conditionally-immortalised cells can be reprogrammed into a pluripotent stem cell phenotype. This provides advantages over existing induced Pluripotent Stem Cells. In particular, despite in vitro immortalisation and long-term culture, it has surprisingly been shown that the neural stem cell lines CTX0E03 and STR0005 can be reprogrammed by exogenous transcription factors. Furthermore, it is also surprising that the conditionally controlled gene remains able to be activated and silenced after the reprogramming, as it was before.

Advantageously, the reprogramming of conditionally-immortalised cells can often be achieved with fewer reprogramming factors than in standard (non conditionally-immortalised) cells. The use of Adult Stem Cells in certain embodiments provides similar advantages.

Furthermore, the conditionally-immortalised nature of the cells provides beneficial controllability over the cells and over the immortalisation system. In some embodiments, these benefits are provided by the C-MYC-ER^{TAM} conditional-immortalisation system. Without wishing to be bound by theory, the use of conditionally-immortalised cells as the source for reprogramming to pluripotency is thought to contribute to the observed benefits over previous attempts using immortalised (i.e. permanently immortalised) cells.

The induced Pluripotent cells of the invention, such as the CTX-iPSCs and STROC-iPSCs in the Examples, represent a very useful clinical resource. They may be differentiated along a desired lineage to generate a target population such as a tissue progenitor cell type or adult stem cell population. Then, provision of the immortalising agent (e.g. 4-OHT) to promote continuous growth and prevent cell cycle exit and associated further differentiation could allow the routine and scalable production of previously-unattainable clinically-relevant subpopulations without repeated cell isolation from primary material or repeating a differentiation protocol de novo from induced pluripotent stem cells each time a new batch of cell therapy product is required. This provides for the possibility of an off-the-shelf cell resource, for example for allogeneic cell therapy.

The ability to re-apply inducible immortalisation to produce at scale an allogeneic, off-the-shelf, adult stem cell therapeutic population, is expected to be particularly beneficial.

Cloning or purification steps can be used to generate pure populations of the desired therapeutic types from more- or less-heterogeneous differentiation cultures for large-scale production of off-the-shelf treatments for conditions for which the original conditionally-immortalised cell (e.g.CTX0E03) itself is unsuitable, avoiding the drawbacks seen in the art with incomplete efficiency of differentiation protocols. This applies to both the cells themselves or microparticle (e.g. exosomal) fractions produced by different cell types with alternative repertoires of payload molecules to those produced by CTX cells themselves.

Furthermore, as these CTX-iPSC-derivative sublines are derived from a cell line which has already passed clinical phase safety trials (CTX), their entry into clinical trials for efficacy in new indications is likely to be accelerated.

In certain aspects, the invention relates to induced pluripotent stem cells generated from different neural stem cells comprising a controllable transgene for conditional immortalisation such as the CTX0E03 or STR0C05 neural stem cell lines derived from cortical and striatal tissue respectively and each from a different human donor, and the progeny of those induced pluripotent stem cells.

### Induction of Pluripotency and iPS Cells

The generation of Induced Pluripotent Cells is known in the art, since Takahashi and Yamanaka showed that stem cells with properties similar to Embryonic Stem Cells could be generated from mouse fibroblasts by simultaneously introducing four genes (Cell. 2006; 126: 663-676). The principle was applied to human cells in 2007 (Takahashi et al Cell. 2007; 131: 861-872; Yu et al Science. 2007; 318: 1917-1920). A recent review is provided by Shi et al, Nature Reviews Drug Discovery volume 16, pages 115-130 (2017).

iPSCs are typically derived by introducing products of specific sets of pluripotency-associated genes, or “reprogramming factors”, into a given cell type. The original set of reprogramming factors (also dubbed Yamanaka factors) are the transcription factors Oct4, Sox2, cMyc, and Klf4.

The generation of iPS cells depends on the transcription factors used for the induction. Oct-3/4 and certain products of the Sox gene family (Sox1, Sox2, Sox3, and Sox15) have been identified as crucial transcriptional regulators involved in the induction process whose absence makes induction impossible. Additional genes, however, including certain members of the Klf family (KIM, Klf2, Klf4, and Klf5), the Myc family (c-myc, L-myc, and N-myc), Nanog, and LIN28, have been identified to increase the induction efficiency.

“POU5F1”, “OCT4” and “OCT3/4” are synonyms for the same transcription factor. This is the transcription factor commonly referred to as OCT4 in the art, but more recently re-named POU5F1 (POU class 5 homeobox 1). These names are used interchangeably herein, as will be apparent to the skilled person.

The reprogramming factors are typically introduced into the cell using viral or episomal vectors, as is well-known in the art. Viral vectors suitable for introducing reprogramming factors into a cell include lentivirus, retrovirus and Sendai-virus. Other techniques for introducing reprogramming factors include mRNA transfection.

Non-integrating reprogramming methods are known in the art, for example as reviewed by Schlaeger et al Nat Biotechnol. 2015 January; 33(1): 58-63. In Sendai-virus reprogramming, Sendai-viral particles are typically used to transduce target cells with replication-competent RNAs that encode the set of reprogramming factors. In Episomal reprogramming, prolonged reprogramming factor expression is typically achieved by Epstein-Barr virus-derived sequences that facilitate episomal plasmid DNA replication in dividing cells. In mRNA reprogramming, cells are typically transfected with in vitro-transcribed mRNAs that encode the reprogramming factors, and chemical measures are often employed to limit activation of the innate immune system by foreign nucleic acids. Owing to the very short half-life of mRNAs, daily transfections are often required to induce hiPSCs.

Transfection of reprogramming factors may be achieved in a variety of ways known in the art, such as by lipofection, nucleofection or electroporation.

In one Example below, conditionally-immortal CTX0E03 cells were reprogrammed to pluripotency using standard non-integrating episomal vectors encoding the “Yamanaka Factors” OCT4, L-MYC, KLF4 and SOX2, and LIN28. In another Example, OCT4 alone is shown to induce pluripotency of CTX0E03. Combinations of transcription factors that were also observed to achieve pluripotency include: OCT4 and SOX2; OCT, KLF4 and SOX2; OCT4, KLF4, SOX2 and MYC.

In certain embodiments, one, two, three or four of OCT4, L-MYC, KLF4 and SOX2, and LIN28 are used to reprogram conditionally-immortalised cells to pluripotency. In certain embodiments, OCT4 and one or more of L-MYC, KLF4 and SOX2, and LIN28 are used. In some embodiments, these factors are used in combination with a cMYC-ER^{TAM} conditional immortalisation system.

In another Example below (Example 3), STR0C05 cells were reprogrammed with the reprogramming plasmids pCE-hOCT3/4, pCE-hSK, pCE-hUL and pCEmP53DD, expressing the transcription factors POU5F1, SOX2, KLF4, L-MYC, LIN28 and a dominant negative inhibitor of p53. Therefore, in certain embodiments the transcription factors for use according to the invention may comprise or consist of POU5F1, SOX2, KLF4, L-MYC, LIN28 and a dominant negative inhibitor of p53. One, two, three or more of these may be removed or replaced as will be apparent to the skilled person. In certain embodiments, one, two, three, four or more of POU5F1, SOX2, KLF4, L-MYC, LIN28 and a dominant negative inhibitor of p53 are used to reprogram conditionally-immortalised cells to pluripotency. In some embodiments, these factors are used in combination with a c-myc-ER^{TAM} conditional immortalisation system.

In some embodiments, MYC activity is provided to promote the reprogramming process by the provision of 4-OHT in the medium to activate a c-myc-ER^{TAM} transgene in the stem cell to be reprogrammed. In certain embodiments, therefore, separately added MYC is not required.

### Conditionally-Immortalised Cells

The invention takes conditionally-immortalised cells and induces them to have a pluripotent phenotype. The conditionally-immortalised cells are typically conditionally-immortalised stem cells, for example conditionally-immortalised adult stem cells.

The conditionally-immortalised cells are typically mammalian, more typically human.

Stem cells are known in the art. Stem cells are cells with the ability to proliferate, exhibit self-maintenance or renewal over the lifetime of the organism and to generate clonally related progeny. The stem cells that are re-programmed according to the invention are typically multipotent cells. The stem cells that are re-programmed according to the invention are typically adult (somatic) stem cells.

The stem cells for use in the invention are isolated. The term “isolated” indicates that the cell or cell population to which it refers is not within its natural environment. The cell or cell population has been substantially separated from surrounding tissue. In some embodiments, the cell or cell population is substantially separated from surrounding tissue if the sample contains at least about 75%, in some embodiments at least about 85%, in some embodiments at least about 90%, and in some embodiments at least about 95% stem cells. In other words, the sample is substantially separated from the surrounding tissue if the sample contains less than about 25%, in some embodiments less than about 15%, and in some embodiments less than about 5% of materials other than the stem cells. Such percentage values refer to percentage by weight. The term encompasses cells which have been removed from the organism from which they originated, and exist in culture. The term also encompasses cells which have been removed from the organism from which they originated, and subsequently re-inserted into an organism. The organism which contains the re-inserted cells may be the same organism from which the cells were removed, or it may be a different organism.

The stem cells are typically allogeneic to any future recipient of the progeny cells produced according to the invention.

The invention uses conditionally-immortalised stem cells, such as a stem cell line, in which the expression of an immortalisation factor can be regulated without adversely affecting the production of therapeutically effective stem cells. This may be achieved by introducing an immortalisation factor which is inactive unless the cell is supplied with an activating agent. Such an immortalisation factor may be a gene such as c-mycER. The c-MycER gene product is a fusion protein comprising a c-Myc variant fused to the ligand-binding domain of a mutant estrogen receptor. C-MycER only drives cell proliferation in the presence of the synthetic steroid 4-hydroxytamoxifen (4-OHT) (Littlewood et al. 1995). This approach allows for controlled expansion of neural stem cells in vitro, while avoiding undesired in vivo effects on host cell proliferation (e.g. tumour formation) due to the presence of c-Myc or the gene encoding it in the neural stem cell line.

In certain embodiments, the conditionally-immortalised stem cell may be:
a mesenchymal stem cell, optionally selected from a bone marrow derived stem cell, an endometrial regenerative cell, a mesenchymal progenitor cell or a multipotent adult progenitor cell;
a neural stem cell;
a haematopoietic stem cell, optionally a CD34+ cell and/or isolated from umbilical cord blood, or optionally a CD34+/CXCR4+ cell;
a non-haematopoietic umbilical cord blood stem cell; or
a mesenchymal stem cell derived from adipose tissue.

In each of these embodiments, the cell is typically mammalian, more typically human.

Typically, the conditionally-immortalised stem cell is a neural stem cell, for example a human neural stem cell.

Neural stem cells give rise to neurons, astrocytes and oligodendrocytes during development and can replace a number of neural cells in the adult brain. Typical neural stem cells for use in certain aspects according to the present invention cells that exhibit one or more of the neural phenotypic markers Musashi-1, Nestin, NeuN, class III β-tubulin, GFAP, NF-L, NF-M, microtubule associated protein (MAP2), S100, CNPase, glypican, (especially glypican 4), neuronal pentraxin II, neuronal PAS 1, neuronal growth associated protein 43, neurite outgrowth extension protein, vimentin, Hu, internexin, 04, myelin basic protein and pleiotrophin, among others.

The neural stem cell may be from a stem cell line, i.e. a culture of stably dividing stem cells. A stem cell line can to be grown in large quantities using a single, defined source.

Preferred conditionally-immortalised neural stem cell lines include the CTX0E03, STR0C05 and HPCOA07 neural stem cell lines, which have been deposited by the applicant of this patent application, ReNeuron Limited, at the European Collection of Animal Cultures (ECACC), Vaccine Research and Production laboratories, Public Health Laboratory Services, Porton Down, Salisbury, Wiltshire, SP4 0JG, with Accession No. 04091601 (CTX0E03); Accession No. 04110301 (STR0C05); and Accession No. 04092302 (HPCOA07). The derivation and provenance of these cells is described in EP1645626 B1 and U.S. Pat. No. 7,416,888, both incorporated herein by reference in their entirety.

### CTX0E03 (ECACC Deposit #04091601)

CTX0E03 is a neural stem cell line in clinical trials as a therapy for ischemic stroke and limb damage. It is controllably immortalised by the integration of a C-MYC-ER^{TAM} fusion protein, which upon binding of the ER^{TAM} domain to the synthetic estrogen derivative 4-hydroxytamoxifen (4-OHT) translocates to the nucleus where the C-MYC domain promotes indefinite cell cycling. Expression of the C-MYC-ER^{TAM} does not apparently affect cell phenotype. Thus an indefinitely-large number of patients may be treated with CTX as an “off-the-shelf” allogeneic therapy. The transgene has been shown to be silenced upon removal of 4-OHT and/or transfer to a patient.

The cells of the CTX0E03 cell line may be cultured in the following culture conditions:
Human Serum Albumin 0.03%
Transferrin, Human 5 μg/ml
Putrescine Dihydrochloride 16.2 μg/ml
Insulin Human recombinant 5 p/ml
Progesterone 60 ng/ml
L-Glutamine 2 mM
Sodium Selenite (selenium) 40 ng/ml

Plus basic Fibroblast Growth Factor (10 ng/ml), epidermal growth factor (20 ng/ml) and 4-hydroxytamoxifen (100 nM) for cell expansion. The cells can be differentiated by removal of the 4-hydroxytamoxifen. Typically, the cells can either be cultured at 5% CO₂/37° C. or under hypoxic conditions of 5%, 4%, 3%, 2% or 1% O₂. These cell lines do not require serum to be cultured successfully. Serum is required for the successful culture of many cell lines, but contains many contaminants. A further advantage of the CTX0E03, STR0C05 or HPCOA07 neural stem cell lines, or any other cell line that does not require serum, is that the contamination by serum is avoided. In some embodiments of the present invention, absence of serum in the system can be maintained, for example by the use of E8 medium for the steps of reprogramming and culture of induced pluripotent stem cells.

CTX culture medium may be supplemented with 4-OHT or not, to provide MYC activity through the c-myc-ER^{TAM} transgene, as desired.

The cells of the CTX0E03 cell line are multipotent cells originally derived from 12 week human fetal cortex. The isolation, manufacture and protocols for the CTX0E03 cell line is described in detail by Sinden, et al. (U.S. Pat. No. 7,416,888 and EP1645626 B1). The CTX0E03 cells are not “embryonic stem cells”, i.e. they are not pluripotent cells derived from the inner cell mass of a blastocyst; isolation of the original cells did not result in the destruction of an embryo. In growth medium CTX0E03 cells are nestin-positive with a low percentage of GFAP positive cells (i.e. the population is negative for GFAP).

CTX0E03 is a clonal cell line that contains a single copy of the c-mycER transgene that was delivered by retroviral infection and is conditionally regulated by 4-OHT (4-hydroxytamoxifen). The C-mycER transgene expresses a fusion protein that stimulates cell proliferation in the presence of 4-OHT and therefore allows controlled expansion when cultured in the presence of 4-OHT. This cell line is clonal, expands rapidly in culture (doubling time 50-60 hours) and has a normal human karyotype (46 XY). It is genetically stable and can be grown in large numbers. The cells are safe and non-tumorigenic. In the absence of growth factors and 4-OHT, the cells undergo growth arrest and differentiate into neurons and astrocytes. Once implanted into an ischemia-damaged brain, these cells migrate only to areas of tissue damage.

The development of the CTX0E03 cell line has allowed the scale-up of a consistent product for clinical use. Production of cells from banked materials allows for the generation of cells in quantities for commercial application (Hodges et al, 2007).

The CTX0E03 drug product can be provided as a fresh (as was the case for the PISCES trial) or frozen suspension of living cells, as described in U.S. Pat. No. 9,265,795 and used in the PISCES II trial. The drug product typically comprises CTX0E03 cells at a passage of

The CTX clinical drug product is typically formulated as an “off the shelf” cryopreserved product in a solvent-free excipient (e.g. as described in U.S. Pat. No. 9,265,795) with a shelf life of many months. This formulation typically comprises Trolox (6-hydroxy-2,5,7,8-tetramethylchroman-2-carboxylic acid), Na⁺, K⁺, Ca²⁺, Mg²⁺, Cl⁻, H₂PO₄⁻, HEPES, lactobionate, sucrose, mannitol, glucose, dextran-40, adenosine and glutathione. One or more, for example two, three, or four, of these excipients may optionally be removed or replaced. Typically, the formulation does not comprise a dipolar aprotic solvent, in particular DMSO.

Clinical release criteria for stem cell products typically include measures of sterility, purity (cell number, cell viability), and a number of other tests of identity, stability, and potency that are required for clinical product release or for information, as requested by regulatory authorities. The tests employed for CTX0E03 are summarised in Table 1, below.

TABLE 1
Identity, Stability, and Potency Tests That Are Employed to Characterize
CTX Cell Banks and/or Drug Products (for Phase II Trial)

| Test | Outcome |
|---|---|
| PCR Sequencing of cDNA | Sequence of insert conforms to transgene identity. |
| | No insertions, deletions, or mutations from |
| | expected sequence |
| Determination of Flanking | Consistent with published sequence |
| Nucleotide Sequence | |
| PCR across integration site | PCR across integration site confirms cell line |
| | identity |
| Karyology | Comparable with published normal chromosome, |
| | male XY |
| Viability and growth | ≥70% viability on recovery. Viable cell numbers at |
| | least double within 7 days |
| c-mycER^{TAM} gene copy number (PCR) | Modal ~1 (range 0.87-3.46) |
| Phenotypic marker (Nestin) | At least 95% of cells are Nestin positive |
| Position, sequence, and indication of | Chromosomal (Chr 13) localization of integrated c- |
| number of integrated target gene by | mycER^{TAM} sequences |
| fluorescent in situ hybridization | |
| Potency | Cell dose-dependent IL-10 production in co-culture |
| | with U937 monocyte cell line |
| Neural differentiation | Upregulation of Tub-β3, GFAP, and GAL-C marker |
| | expression by qPCR after seeding into Alvatex ® |
| | three-dimensional cell matrix |

The CTX0E03 cell line has been previously demonstrated, using a human PBMC assay, not to be immunogenic. The lack of immunogenicity allows the cells to avoid clearance by the host/patient immune system and thereby exert their therapeutic effect without a deleterious immune and inflammatory response.

Pollock et al 2006 describes that transplantation of CTX0E03 in a rat model of stroke (MCAo) caused statistically significant improvements in both sensorimotor function and gross motor asymmetry at 6-12 weeks post-grafting. These data indicate that CTX0E03 has the appropriate biological and manufacturing characteristics necessary for development as a therapeutic cell line.

Stevanato et al 2009 confirms that CTX0E03 cells downregulated c-mycERTAM transgene expression both in vitro following EGF, bFGF and 4-OHT withdrawal and in vivo following implantation in MCAo rat brain. The silencing of the c-mycER^{TAM} transgene in vivo provides an additional safety feature of CTX0E03 cells for potential clinical application.

Smith et a/2012 describe preclinical efficacy testing of CTX0E03 in a rat model of stroke (transient middle cerebral artery occlusion). The results indicate that CTX0E03 implants robustly recover behavioural dysfunction over a 3 month time frame and that this effect is specific to their site of implantation. Lesion topology is potentially an important factor in the recovery, with a stroke confined to the striatum showing a better outcome compared to a larger area of damage.

### STR0005 (ECACC Deposit #04110301)

This c-MycER^{TAM} transduced-neural stem cell line was derived from 12 week fetal striatum. The line is maintained on laminin coated culture flasks using defined serum free “Human Media” in the presence of bFGF, EGF and 4-hydroxy tamoxifen. In routine culture the cell line has a doubling time of 3-4 days although in short term culture a doubling time of 20-30 h was seen.

In growth medium the cells are nestin-positive, beta-III tubulin-negative with a low percentage of GFAP positive cells. Following differentiation for 7 days there is down regulation of nestin with low-level expression of beta III tubulin and strong expression of GFAP suggesting that the cell line becomes predominantly astrocytic.

This cell line is genetically normal, male XY, and stable over 50 population doublings.

The lines described here were derived under Quality Assured conditions suitable for progressing designated lines for clinical use. As source material, human neural stem cells were isolated post mortem from the striatum of a 12-week gestation fetus GS006 by enzymatic digestion with trypsin in combination with mechanical trituration. Once established in culture these primary neural cells were transformed by retroviral transduction with the c-MycERTAM oncogene (as described for the CTXOEO3 cell line above) and a range of clonal and mixed population cell lines isolated. All lines in this series were derived on laminin coated culture-ware and using Human Media (HM); DMEM:F12 plus designated supplements as described below.

### Human Media (HM)

DMEM:F12 supplemented with the components listed below:

Human Serum Albumin 0.03%.

Transferrin, Human 100 μg/ml.

Putrescine Dihydrochloride 16.2 μg/ml.

Insulin, Human recombinant 5 μg/ml.

L-Thyroxine (T4) 400 ng/ml.

Tri-Iodo-Thyronine (T3) 337 ng/ml.

Progesterone 60 ng/ml.

L-Glutamine 2 mM.

Sodium Selenite (selenium) 40 ng/ml.

Heparin, sodium salt 10 Units/ml.

Corticosterone 40 ng/ml.

Plus basic Fibroblast Growth Factor (10 ng/ml) and epidermal growth factor (20 ng/ml) for cell expansion.

### STR0005 Growth Characteristics

Under routine culture conditions cells are expanded from frozen stocks, usually 2-4 million cells in T180 culture flasks After several media changes the cells are passaged when confluent. From process records, population doubling times for STR0C05 have been estimated at 3-4 days as shown on the graph below. This doubling time is slower than for log phase growth and also includes cell loss during the passaging.

As a more representative assessment of log phase growth for STR0C05, a cell proliferation assay was set up using the Cyquant fluorescent dye (Molecular Probes). Cell number is measured using a Tecan Magellan fluorescence plate reader; ex . . . 480 nm; em 520 nm.

STR0C05 cells were passaged, resuspended in HM plus growth factors and seeded on laminin coated 96 well strip-well plates at 5000 cells/well. A time course study was carried out by removing strips from the plate on a daily basis, n=16 wells per time point, removing the media and freezing the cells at −70° C.

At the end of the time course all the frozen strips were put back together on the plate and analysed with the Cyquant assay. Briefly cells are lysed in lysis buffer then Cyquant reagent added and placed in dark for 5 minutes. A 150 ul sample of each well was then transferred to black, Optilux plates for reading on a Tecan Magellan plate reader. Data was exported to an Excel spreadsheet for numerical averaging and further exported to GraphPad Prism for analysis.

The results showed that the cells grew steadily over 7 days with an estimated doubling time of 20-30 hours.

### STR0005 Phenotype

The phenotype of the STR0C05 has been profiled using immunocytochemistry to stain for the neural stem cell marker nestin and to stain for mature markers of differentiation, beta-Ill tubulin (neuronal) and GFAP (astrocytic).

STR0C05 phenotype was determined in the presence and absence of growth factors plus 4-OHT. Cells were originally sourced from STR0005 working stock. Cells were passaged and seeded in 96 well plates.

Cells were fixed in 4% paraformaldehyde for 15 minutes at room temperature, washed with PBS and permeabilised with 0.1% Triton X100/PBS for 15 minutes. Non-specific binding was then blocked with 10% Normal Goat Serum (NGS) in PBS for 1 hour at room temperature. Cells were then probed with antibodies to Nestin (1:200, Chemicon), Beta-III Tubulin (1:500; Sigma) and GFAP (1:5000; DAKO) at room temperature overnight. After washing with PBS, they were then processed with filtered Alexa Goat a Mouse 488 (1:200; Molecular Probes) and Alexa Goat a Rabbit 568 (1:2500; Molecular Probes) dissolved in 1% NGS/PBS for 1 hour at room temperature. They were then washed with PBS and counterstained with Hoechst 33342 (Sigma) for 2 minutes before being analysed on a fluorescent microscope.

Removal of growth factors and 4-OHT from the medium induces a morphological and phenotypic change in the cells that is accompanied by down regulation of nestin. Specifically a small proportion of the cells become positive for the neuronal marker beta-III tubulin and acquire a neuronal morphology with rounded cell bodies extending into dendritic/axonal outgrowths. The more dominant phenotypic change however is the up-regulation of GFAP suggesting a predominance of an astrocytic lineage.

### Clonality

### Southern Blot for STR0005

In two separate experiments there is no evidence of probe hybridisation in contrast to clear bands seen with other cell lines.

### Cell Populations

The invention uses and relates to a population of isolated stem cells, wherein the population essentially comprises only stem cells of the invention, i.e. the stem cell population is substantially pure. In many aspects, the stem cell population comprises at least about 75%, or at least 80% (in other aspects at least 85%, 90%, 91%, 92%, 93%, 94%, 95%, 96%, 97%, 98%, 99%, 99.5%, 99.9% or 100%) of the stem cells of the invention, with respect to other cells that make up a total cell population. For example, with respect to neural stem cell populations, this term means that there are at least about 75%, in some embodiments at least about 85%, in some embodiments at least about 90%, and in some embodiments at least about 95% pure, neural stem cells compared to other cells that make up a total cell population. The term “substantially pure” therefore refers to a population of stem cells of the present invention that contain fewer than about 25%, in some embodiments fewer than about 15%, and in some embodiments fewer than about 5%, of cells that are not neural stem cells.

Isolated stem cells can be characterised by a distinctive expression profile for certain markers and is distinguished from stem cells of other cell types. When a marker is described herein, its presence or absence may be used to distinguish the neural stem cell.

A neural stem cell population may in some embodiments be characterised in that the cells of the population express one, two, three, four, five or more, for example all, of the markers Nestin, Sox2, GFAP, βIII tubulin, DCX, GALC, TUBB3, GDNF and IDO.

Typically, neural stem cells are nestin positive.

A “Marker” refers to a biological molecule whose presence, concentration, activity, or phosphorylation state may be detected and used to identify the phenotype of a cell.

A stem cell of the invention is typically considered to carry a marker if at least about 70% of the cells of the population show a detectable level of the marker. In other aspects, at least about 80%, at least about 90% or at least about 95% or at least about 97% or at least about 98% or more of the population show a detectable level of the marker. In certain aspects, at least about 99% or 100% of the population show detectable level of the markers. Quantification of the marker may be detected through the use of a quantitative RT-PCR (qRT-PCR) or through fluorescence activated cell sorting (FACS). It should be appreciated that this list is provided by way of example only, and is not intended to be limiting. Typically, a neural stem cell of the invention is considered to carry a marker if at least about 90% of the cells of the population show a detectable level of the marker as detected by FACS.

The term “expressed” is used to describe the presence of a marker within a cell. In order to be considered as being expressed, a marker must be present at a detectable level. By “detectable level” is meant that the marker can be detected using one of the standard laboratory methodologies such as qRT-PCR, or RT-PCR, blotting, Mass Spectrometry or FACS analysis. A gene is considered to be expressed by a cell of the population of the invention if expression can be reasonably detected at a crossing point (cp) values below or equal 35 (standard cut off on a qRT-PCR array). The Cp represents the point where the amplification curve crosses the detection threshold, and can also be reported as crossing threshold (ct).

The terms “express” and “expression” have corresponding meanings. At an expression level below this cp value, a marker is considered not to be expressed. The comparison between the expression level of a marker in a stem cell of the invention, and the expression level of the same marker in another cell, such as for example an mesenchymal stem cell, may preferably be conducted by comparing the two cell types that have been isolated from the same species. Preferably this species is a mammal, and more preferably this species is human. Such comparison may conveniently be conducted using a reverse transcriptase polymerase chain reaction (RT-PCR) experiment.

As used herein, the term “significant expression” or its equivalent terms “positive” and “+” when used in regard to a marker shall be taken to mean that, in a cell population, more than 20%, preferably more than, 30%, 40%, 50%, 60%, 70%, 80%, 90% 95%, 98%, 99% or even all of the cells of the cells express said marker.

As used herein, “negative” or “−” as used with respect to markers shall be taken to mean that, in a cell population, fewer than 20%, 10%, preferably fewer than 9%, 8%, 7%, 6%, 5%, 4%, 3%, 2%, 1% or none of the cells express said marker.

Expression of cell surface markers may be determined, for example, by means of flow cytometry and/or Fluorescence activated cell sorting (FACS) for a specific cell surface marker using conventional methods and apparatus (for example a Beckman Coulter Epics XL FACS system used with commercially available antibodies and standard protocols known in the art) to determine whether the signal for a specific cell surface marker is greater than a background signal. The background signal is defined as the signal intensity generated by a non-specific antibody of the same isotype as the specific antibody used to detect each surface marker. For a marker to be considered positive the specific signal observed is typically more than 20%, preferably stronger than 30%, 40%, 50%, 60%, 70%, 80%, 90%, 100%, 500%, 1000%, 5000%, 10000% or above, greater relative to the background signal intensity. Alternative methods for analysing expression of cell surface markers of interest include visual analysis by electron microscopy using antibodies against cell-surface markers of interest.

### Stem Cell Culture and Production

Simple bioreactors for stem cell culture are single compartment flasks, such as the commonly-used T-175 flask (e.g. the BD Falcon™ 175 cm² Cell Culture Flask, 750 ml, tissue-culture treated polystyrene, straight neck, blue plug-seal screw cap, BD product code 353028).

The conditionally-immortalised stem cells may typically be taken from proliferating stem cells cultured in T-175 or T-500 flasks.

Bioreactors can also have multiple compartments, as is known in the art. These multi-compartment bioreactors typically contain at least two compartments separated by one or more membranes or barriers that separate the compartment containing the cells from one or more compartments containing gas and/or culture medium. Multi-compartment bioreactors are well-known in the art. An example of a multi-compartment bioreactor is the Integra CeLLine bioreactor, which contains a medium compartment and a cell compartment separated by means of a 10 kDa semi-permeable membrane; this membrane allows a continuous diffusion of nutrients into the cell compartment with a concurrent removal of any inhibitory waste product. The individual accessibility of the compartments allows to supply the cells with fresh medium without mechanically interfering with the culture. A silicone membrane forms the cell compartment base and provides an optimal oxygen supply and control of carbon dioxide levels by providing a short diffusion pathway to the cell compartment. Any multi-compartment bioreactor may be used according to the invention.

The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium”. “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the invention while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

Microparticles Produced by the Pluripotent Cells of the Invention and their Progeny

The pluripotent stem cells of the invention, and the differentiated cells generated from those cells, will produce microparticles. The invention provides, in one aspect, microparticles obtainable from the induced pluripotent stem cells of the invention, or from differentiated cells generated from those iPS cells. These microparticles can be used in therapy.

The microparticles obtained from cells of the invention can also be used as delivery vehicles for exogenous cargo. The cargo may, in some embodiments, be exogenous nucleic acid (e.g. DNA or RNA, in particular an RNAi agent such as siRNA or chemically-modified siRNA), exogenous protein (e.g. an antibody or antibody fragment, a signalling protein, or a protein drug). It is known in that art that cargo can be directly loaded into microparticles, for example by transfection or electroporation. It is also known that manipulating the cell that produces the microparticle can change the content of the microparticle.

The nature, content and characteristics of microparticles are influenced by the cell that produces them. Therefore, the invention advantageously provides for a diverse range of microparticles to be produced from a single well-characterised starting material (i.e. the conditionally-immortalised cell). For example, microparticles can be isolated from the iPS cell or any more differentiated cell derived from that cell, such as a cell that has entered the endoderm, mesoderm or ectoderm lineage. This allows for the provision of many different microparticles from a single, known starting cell.

A “microparticle” is an extracellular vesicle of 30 to 1000 nm diameter that is released from a cell. It is limited by a lipid bilayer that encloses biological molecules. The term “microparticle” is known in the art and encompasses a number of different species of microparticle, including a membrane particle, membrane vesicle, microvesicle, exosome-like vesicle, exosome, ectosome-like vesicle, ectosome or exovesicle. The different types of microparticle are distinguished based on diameter, subcellular origin, their density in sucrose, shape, sedimentation rate, lipid composition, protein markers and mode of secretion (i.e. following a signal (inducible) or spontaneously (constitutive)). Four of the common microparticles and their distinguishing features are described in Table 1, below. In certain embodiments, the microparticle is an exosome.

TABLE 1
Various Microparticles

| Microparticle | Typical Size | Shape | Markers | Lipids | Origin |
|---|---|---|---|---|---|
| Microvesicles | 100-1000 nm | Irregular | Integrins, | Phosphatidylserine | Plasma |
| | | | selectins, | | membrane |
| | | | CD40 ligand | | |
| Exosome-like | 20-50 nm | Irregular | TNFRI | No lipid rafts | MVB from |
| vesicles | | | | | other |
| | | | | | organelles |
| Exosomes | 30-100 nm; | Cup | Tetraspanins | Cholesterol, | Multivesicular |
| | (<200 nm) | shaped | (e.g. CD63, CD9), | sphingomyelin, | endosomes |
| | | | Alix, TSG101, ESCRT | ceramide, lipid rafts, | |
| | | | | phosphatidylserine | |
| Membrane | 50-80 nm | Round | CD133, | Unknown | Plasma |
| particles | | | no CD63 | | membrane |

Microparticles are thought to play a role in intercellular communication by acting as vehicles between a donor and recipient cell through direct and indirect mechanisms. Direct mechanisms include the uptake of the microparticle and its donor cell-derived components (such as proteins, lipids or nucleic acids) by the recipient cell, the components having a biological activity in the recipient cell. Indirect mechanisms include microvesicle-recipient cell surface interaction, and causing modulation of intracellular signalling of the recipient cell. Hence, microparticles may mediate the acquisition of one or more donor cell-derived properties by the recipient cell. It has been observed that, despite the efficacy of stem cell therapies in animal models, the stem cells do not appear to engraft into the host. Accordingly, the mechanism by which stem cell therapies are effective is not clear. Without wishing to be bound by theory, the inventors believe that the microparticles secreted by neural stem cells play a role in the therapeutic utility of these cells and are therefore therapeutically useful themselves.

The microparticles of the invention are isolated, as defined herein for the cells.

The invention provides a population of isolated stem cell microparticles produced by a cell of the invention, wherein the population essentially comprises only microparticles of the invention, i.e. the microparticle population is pure. In many aspects, the microparticle population comprises at least about 80% (in other aspects at least 85%, 90%, 91%, 92%, 93%, 94%, 95%, 96%, 97%, 98%, 99%, 99.5%, 99.9% or 100%) of the microparticles of the invention.

In certain embodiments, the microparticles are exosomes. The lipid bilayer of an exosome is typically enriched with cholesterol, sphingomyelin and ceramide. Exosomes also express one or more tetraspanin marker proteins. Tetraspanins include CD81, CD63, CD9, CD53, CD82 and CD37. CD63 is a typical exosome marker. Exosomes can also include growth factors, cytokines and RNA, in particular miRNA. Exosomes typically express one or more of the markers TSG101, Alix, CD109, thy-1 and CD133. Alix (Uniprot accession No. Q8WUM4), TSG101 (Uniprot accession No. Q99816) and the tetraspanin proteins 0081 (Uniprot accession No. P60033) and CD9 (Uniprot accession No. P21926) are characteristic exosome markers.

Alix is an endosomal pathway marker. Exosomes are endosomal-derived and, accordingly, a microparticle positive for this marker is characterised as an exosome. Exosomes of the invention are typically positive for Alix. Microvesicles are typically negative for Alix.

In some embodiments, the microparticles such as exosomes can be loaded with exogenous cargo. The exogenous cargo can be a protein (for example an antibody), peptide, drug, prodrug, hormone, diagnostic agent, nucleic acid (e.g. RNAi agent such as miRNA, siRNA or shRNA, or a DNA or RNA vector), carbohydrate or other molecule of interest. The cargo can be loaded directly into the exosomes, for example by electroporation or transfection, or can be loaded into the exosome by engineering the cell that produces the exosome such that the cell encapsulates the cargo into the exosome before exosome release. The loading of cargo into microparticles such as exosomes is known in the art.

### Pharmaceutical Compositions

The pluripotent stem cells of the invention can be differentiated to generate cells that are useful in therapy and can therefore be formulated as a pharmaceutical composition. The pluripotent stem cells of the invention, and the differentiated cells generated from those cells, will produce microparticles as described elsewhere herein, that may also be useful in therapy and can therefore be formulated as a pharmaceutical composition.

A pharmaceutically acceptable composition typically includes at least one pharmaceutically acceptable carrier, diluent, vehicle and/or excipient in addition to the therapeutic cells or microparticles. An example of a suitable carrier is Ringer's Lactate solution. A thorough discussion of such components is provided in Gennaro (2000) Remington: The Science and Practice of Pharmacy, 20th edition, ISBN: 0683306472.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition, if desired, can also contain minor amounts of pH buffering agents. The composition may comprise storage media such as Hypothermosol®, commercially available from BioLife Solutions Inc., USA. Examples of suitable pharmaceutical carriers are described in “Remington's Pharmaceutical Sciences” by E W Martin. Such compositions will contain a prophylactically or therapeutically effective amount of a prophylactic or therapeutic stem cell preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, semi-solid, and liquid dosage forms, such as lyophilized preparations, frozen preparations, liquid solutions or suspensions, injectable and infusible solutions. The pharmaceutical composition is preferably injectable.

Pharmaceutical compositions will generally be in aqueous form. Compositions may include a preservative and/or an antioxidant.

To control tonicity, the pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition is preferably sterile. The composition is preferably non-pyrogenic.

In a typical embodiment, the cells or microparticles are suspended in a composition comprising 1, 2, 3, 4, 5, 6, 7, 8, 9, 10, or more excipients selected from 6-hydroxy-2,5,7,8-tetramethylchroman-2-carboxylic acid (Trolox®), Na⁺, K⁺, Ca²⁺, Mg²⁺, Cl⁻, H₂PO₄⁻, HEPES, lactobionate, sucrose, mannitol, glucose, dextron-40, adenosine and glutathione. In one embodiment the composition comprises all of these excipients. Typically, the composition will not include a dipolar aprotic solvent, e.g. DMSO. Suitable compositions are available commercially, e.g. HypoThermasol®-FRS. Such compositions are advantageous as they allow the cells to be stored at 4° C. to 25° C. for extended periods (hours to days) or preserved at cryothermic temperatures, i.e. temperatures below −20° C. The stem cells may then be administered in this composition after thawing.

Although the invention has been described in detail for purposes of clarity of understanding, certain modifications may be practiced within the scope of the appended claims. All publications, accession numbers, and patent documents cited in this application are hereby incorporated by reference in their entirety for all purposes to the same extent as if each were so individually denoted. To the extent more than one sequence is associated with an accession number at different times, the sequences associated with the accession number as of the effective filing date of this application is meant. The effective filing date is the date of the earliest priority application disclosing the accession number in question. Unless otherwise apparent from the context any element, embodiment, step, feature or aspect of the invention can be performed in combination with any other.

The invention is further described with reference to the following non-limiting examples. In these examples, the inventors first demonstrate that conditional immortalised neural stem cells (CTX0E03; deposited on 16 Sep. 2004 by the applicant of this patent application, ReNeuron Limited, at the European Collection of Animal Cultures (ECACC) with Accession No. 04091601) can be reprogrammed to pluripotency, on the basis of several independent replicates. These iPSCs are then differentiated into Mesenchymal stem cells (MSCs). The genetic reprogramming and pluripotency of the CTX-iPSCs is also confirmed.

The inventors then demonstrate the successful reprogramming of another conditionally immortalised adult stem cell type. This line is STR0005, derived from fetal striatal cells (and deposited on 3 Nov. 2004 by the applicant of this patent application, ReNeuron Limited, at the European Collection of Animal Cultures (ECACC) with Accession No. 04110301). Generation of iPSCs from STR0005 and subsequent differentiation of these STROC-iPSCs to endoderm, mesoderm and ectoderm lineages is shown. These data confirm that the benefits afforded by the invention are not limited to the CTX cell line in which we have first demonstrated it, but apply widely, to any conditionally-immortalised adult cell type.

The Examples then provide a further characterisation of the MSC cells derived from the reprogrammed iPSCs, reinforcing the finding that it is possible to expand an adult stem cell type derived from these iPSCs beyond the normal limits for such cells, thereby permitting the treatment of large numbers of patients from such a line. These CTX-iPSC-MSCs are shown (FIG. 10) to differentiate into cartilage (shown by alcian blue staining of sialoglycans), fat (shown by staining of intracellular lipid droplets with oil red 0) and bone (shown by alizarin red staining of deposited calcium) cells.

Finally, yet further detailed characterisation of the CTX-iPSC cells is provided.

### EXAMPLES

### Example 1: iPSCs Derived from Inducibly-Immortalised Adult Stem Cells as a Source for Clinical-Scale Manufacture of Allogeneic Cell Therapies

### Introduction

Induced pluripotent stem cells (iPSCs) have great potential as a source material for cell therapies
Candidate therapeutic populations are typically adult stem cells or tissue progenitors (ASCs/TPs) rather than terminally-differentiated cells
ASCs/TPs are often difficult to culture and purify
Conditional immortalisation of ASCs/TPs would be beneficial for the scalable production of cells for allogeneic cell therapy
CTX is a neural stem cell line in clinical trials for ischemic stroke. It is immortalised with a c-myc-ER^{TAM} transgene, controllable by the addition of 4-hydroxytamoxifen (4-OHT) to the culture medium

### Reprogramming CTX0E03 to Pluripotency

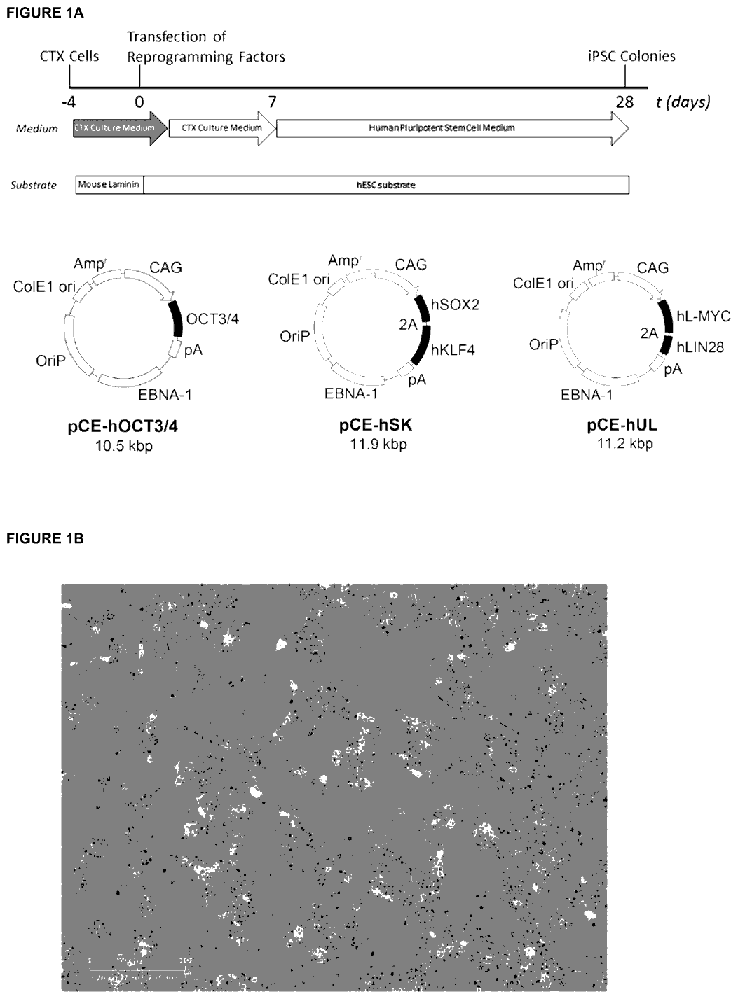

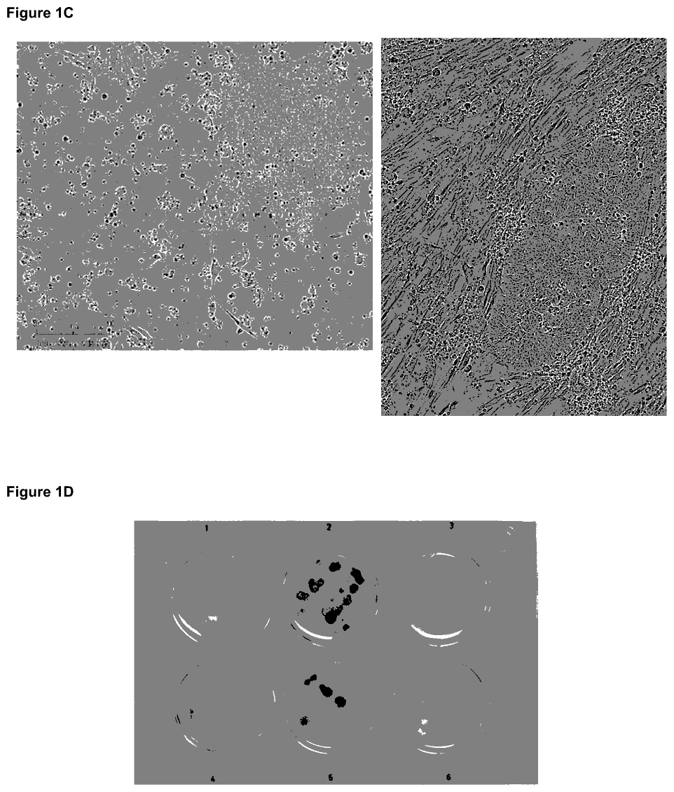

CTX0E03 cells were reprogrammed to pluripotency using standard non-integrating episomal vectors encoding the “Yamanaka Factors” (OCT4, L-MYC, KLF4 and SOX2, “OKSM”, and LIN28) (FIG. 1).

The CTX cells were successfully reprogrammed, independently, several times.

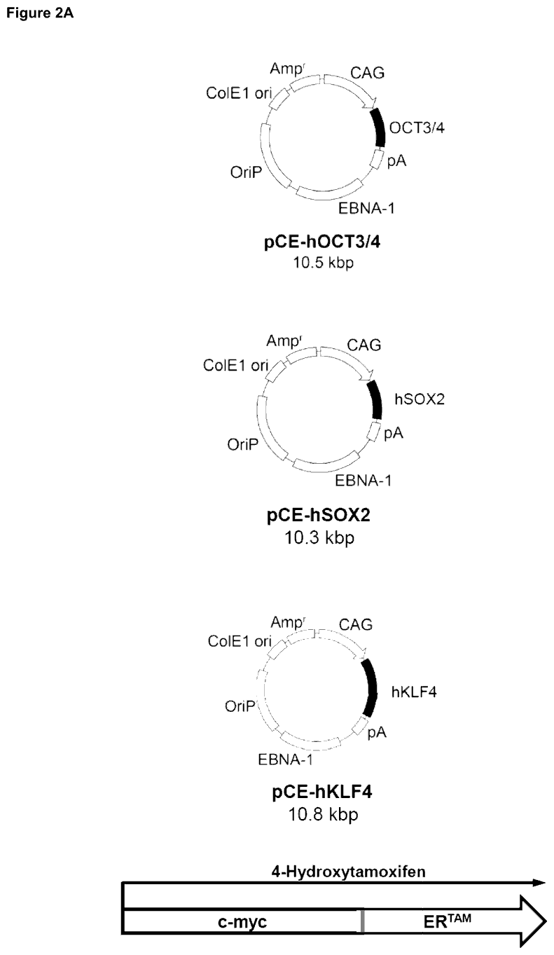

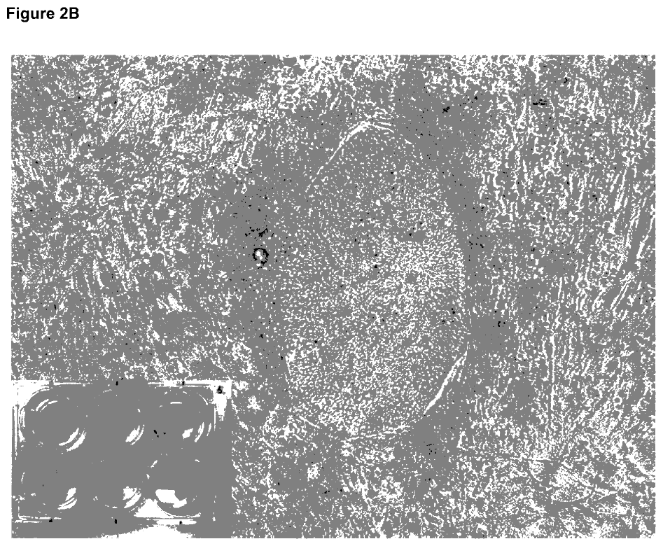

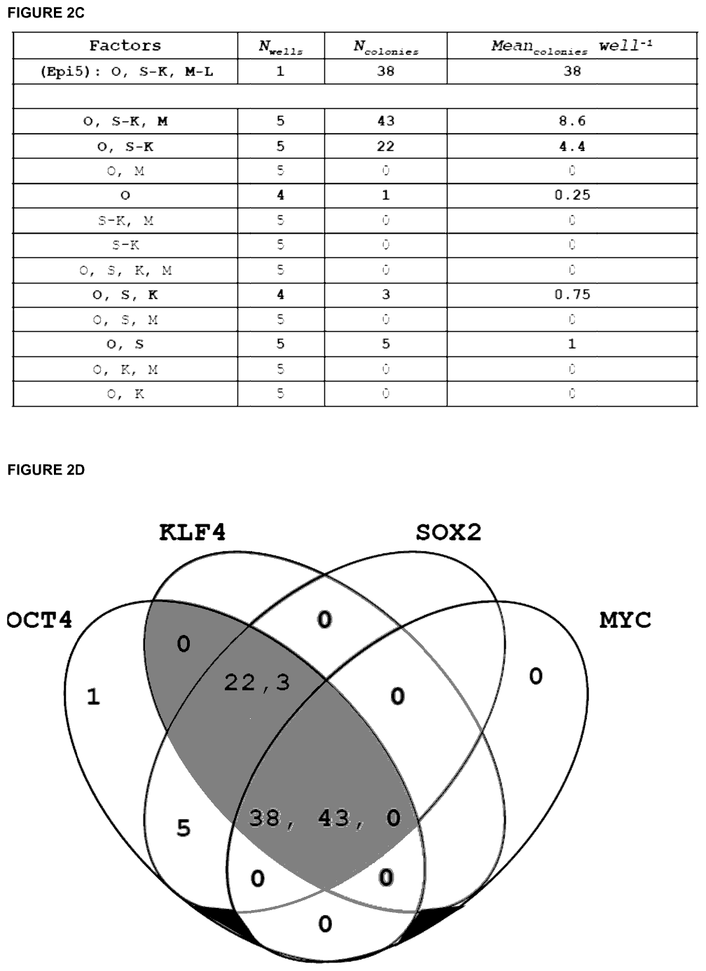

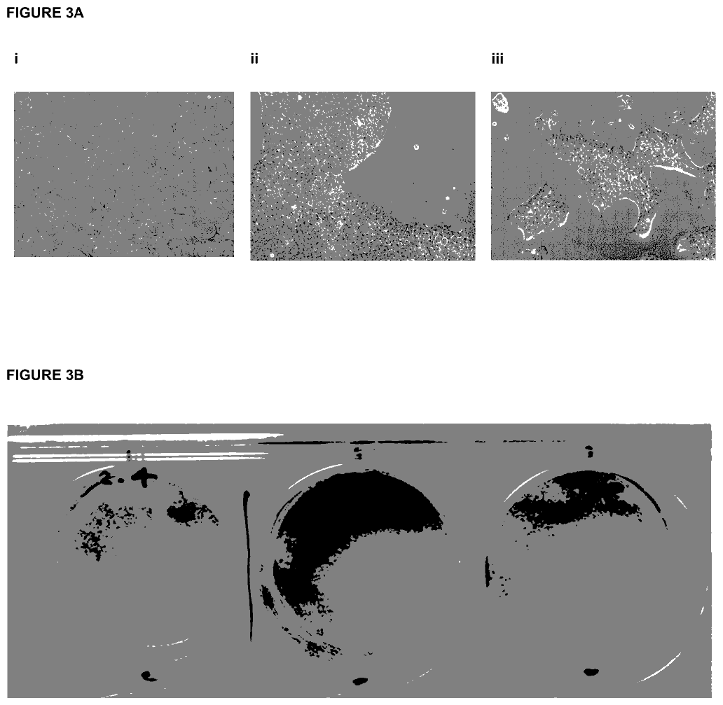

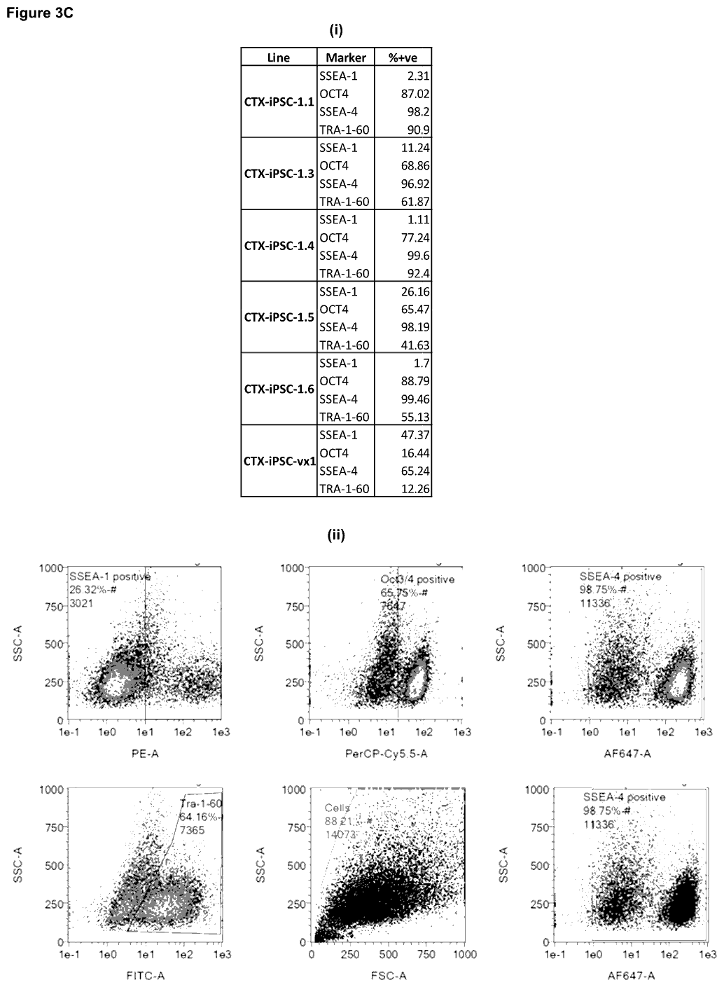

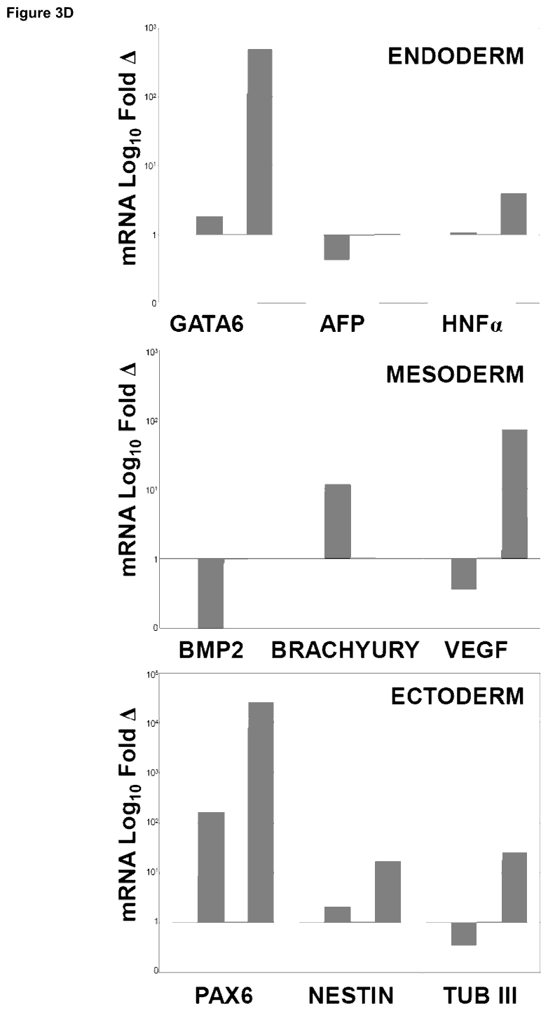

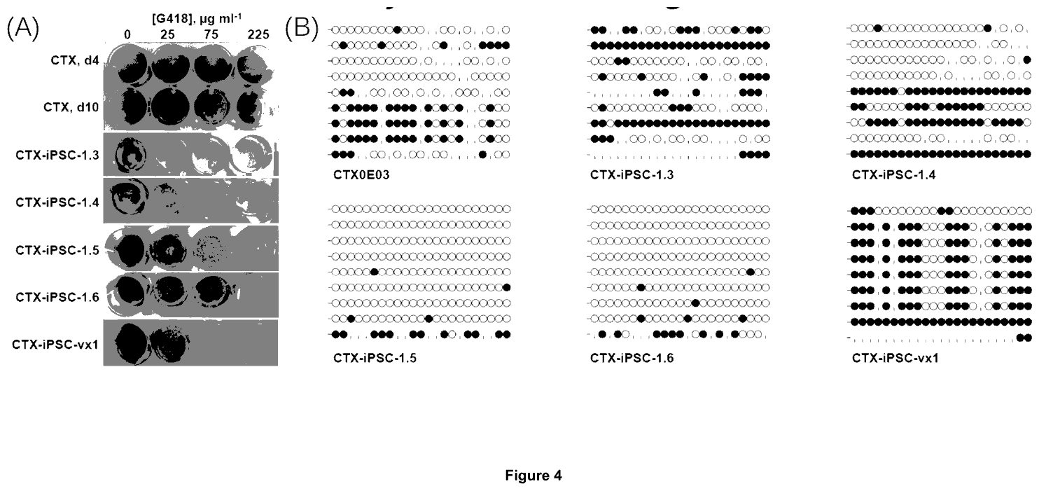

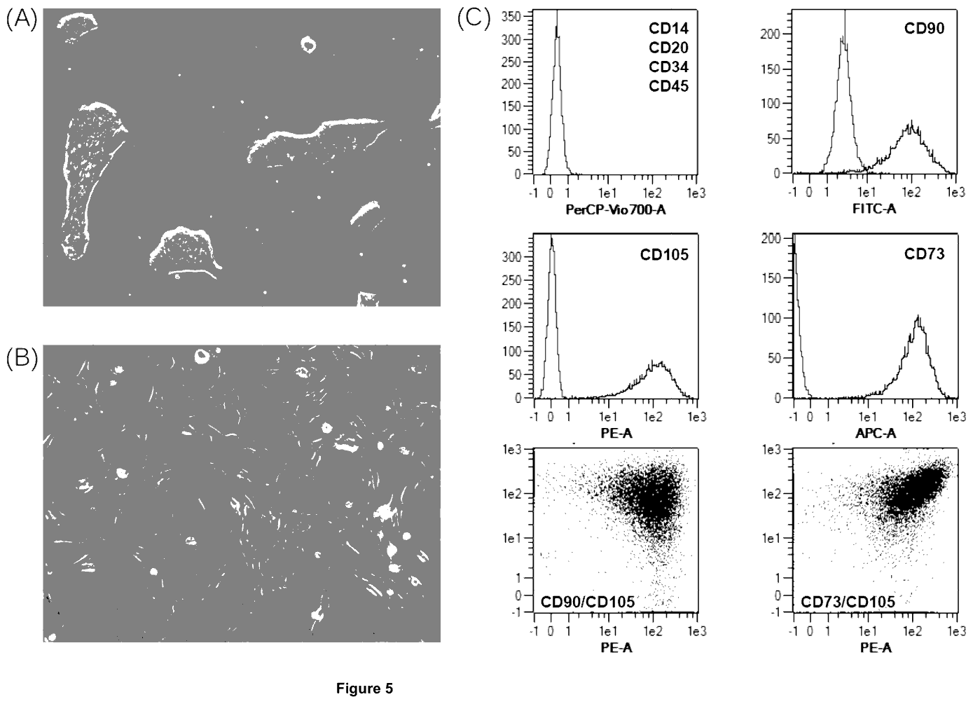

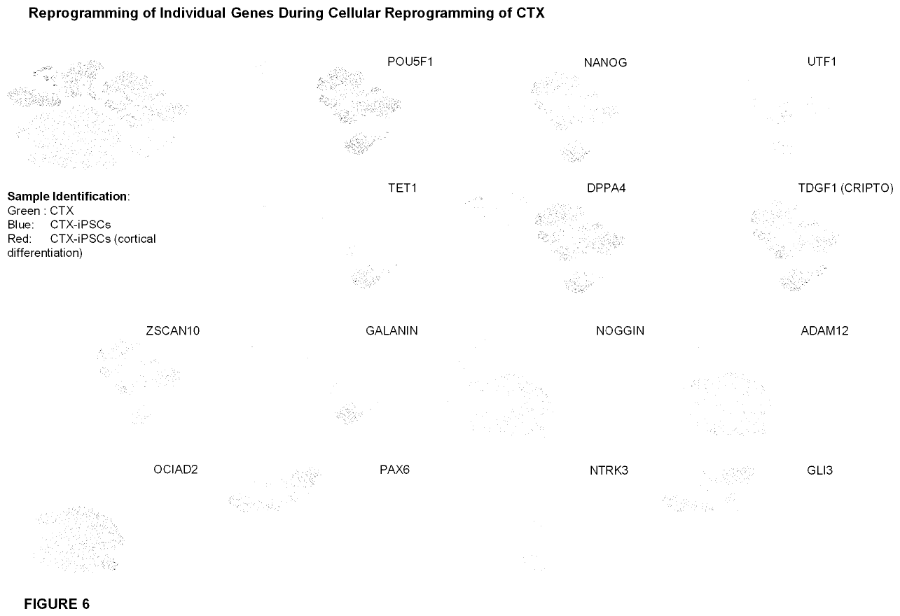

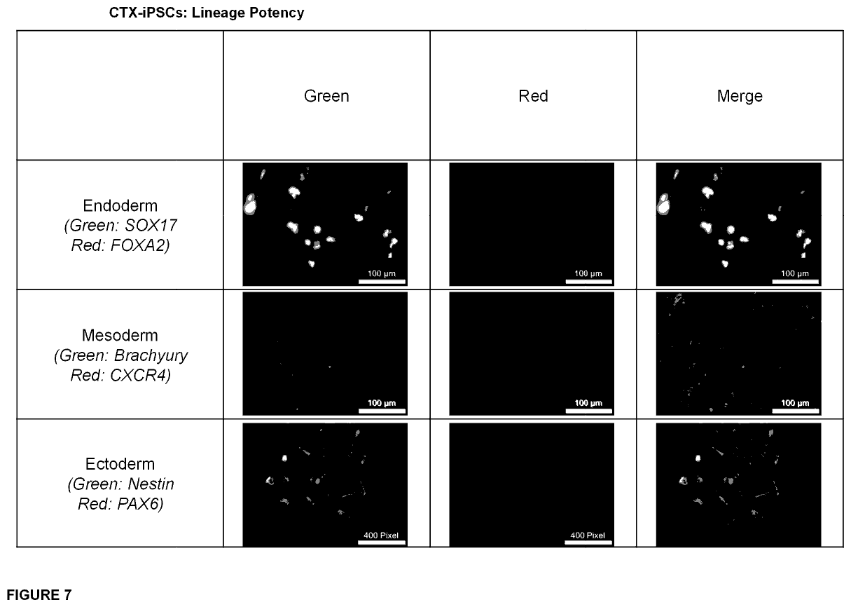

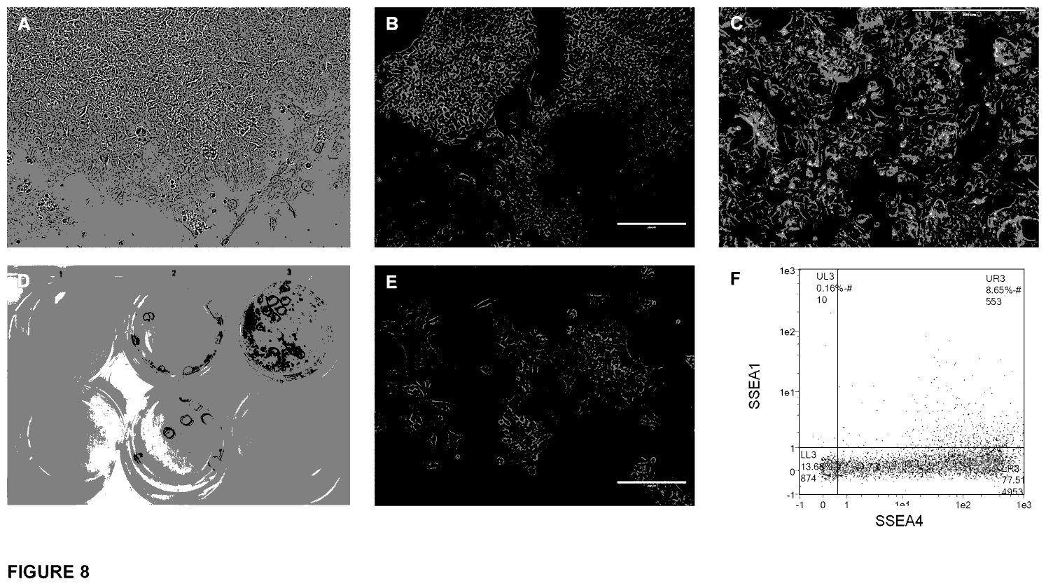

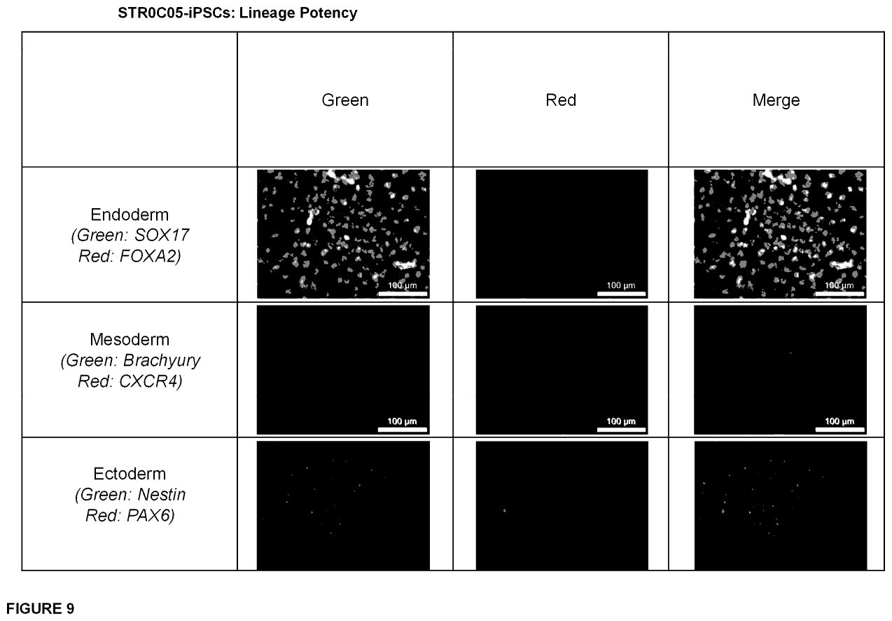

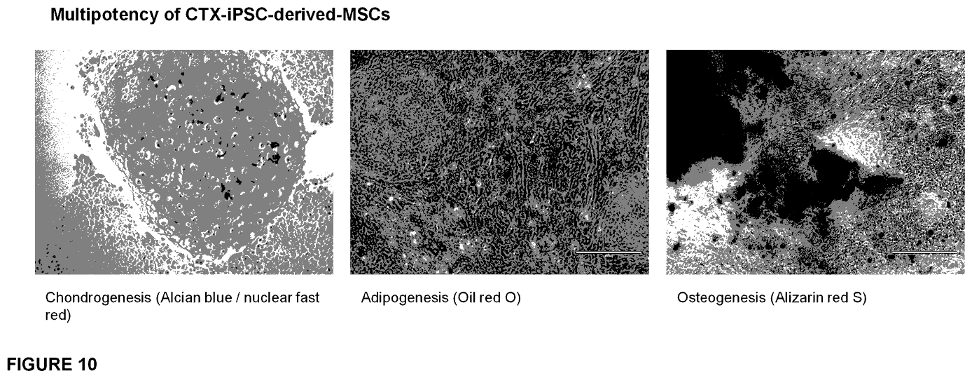

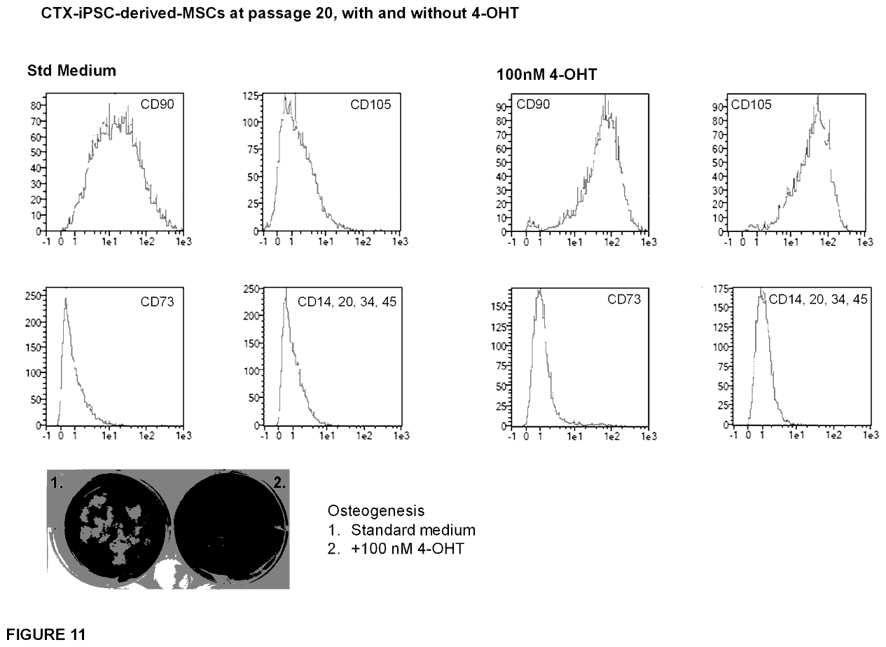

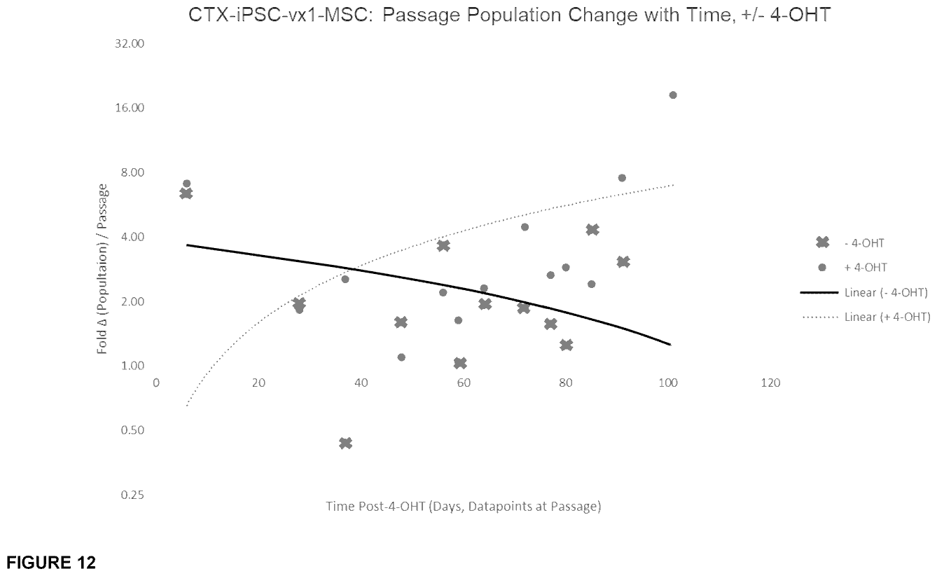

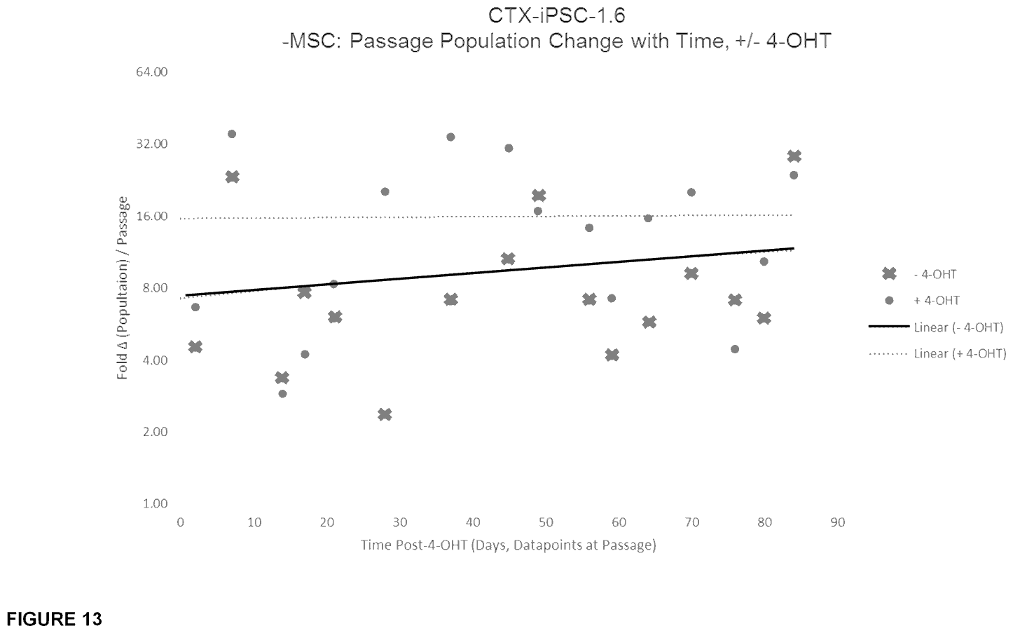

CTX-iPSCs share many features characteristic of human iPSCs and ESCs. After reprogramming, cell morphology changes dramatically from the neuronal phenotype with extended processes characteristic of CTX cells to one of small, rounded, undifferentiated cells with prominent nucleoli and difficult-to-distinguish divisions between cells densely packed into “islands” characteristic of human pluripotent stem cells (FIG. 10, FIG. 2). CTX-iPSCs express the tissue non-specific alkaline phosphatase enzymatic marker at day 21 endpoint (FIG. 1D, FIG. 3).

### Varying Transcription Factor Combinations to Dissect CTX Reprogramming Requirements.

FIG. 2 shows that CTX0E03 cells are reprogrammable with fewer factors. (A) Vectors expressing single factors, pCE-OCT3/4, pCE-SOX2 and pCE-KLF4; 4-OHT provision mimics MYC via c-myc-ER^{TAM}. (B) Inset: example AP-stained plate for colony counting. Main image: colony reprogrammed with transcription factor OCT4 alone. (C) Colony numbers obtained with different factor combinations (S-K: pCE-SK, M-L: pCE-UL, S: pCE-50X2, K: pCE-KLF4, M: 4-OHT→d 14). (D) Venn diagram showing combination effects (numbers: x colonies obtained; zeroes: no colonies).

CTX-iPSCs Share Many Features with Classical hPSCs

The pluripotent phenotype of the CTX-iPSCs is shown in FIG. 3.

(A) Cell and colony morphology assessment in of CTX-iPSCs on two different cell lines (ii, iii) derived from CTX cells by reprogramming to pluripotency by transfection of the OKSML transcription factor set, shows that these reprogrammed cell lines recapitulate the dense colonies of small, closely-packed cells with prominent nucleoli characteristic of hPSCs, differing markedly from the neuronal phenotype of the parental CTX cells (i).

CTX-iPSC lines express the enzymatic marker alkaline phosphatase (pink stain), as shown in FIG. 3B.

As expected for human pluripotent stem cells, flow cytometry shows that CTX-iPSCs are positive for the canonical pluripotent transcription factor OCT4, and the cell surface antigens TRA-1-60 and SSEA-4, but do not express the early differentiation marker SSEA-1. (FIG. 3C).

(D) RT-qPCR showing upregulation of lineage-specific markers upon in vitro differentiation to endoderm, mesoderm and ectoderm (individual CTX-iPSC lines indicated by shade).

Status of the c-Myc-ERTAM Transgene in CTX-iPSCs

Assessment of the transgene locus in CTX-iPSCs is shown in FIG. 4.

(A) Giemsa staining of parental CTX0E03 cells (top, 4 days, 2nd row, 10 days) and five CTX-iPSC lines at 4 days in G418 (3rd-7th rows) indicates expression activity of the c-myc-ER^{TAM}-associated NeoR gene.

(B) Bisulphite-conversion of the CMV-IE promoter driving the c-myc-ER^{TAM} transgene shows the cytosine methylation state at the locus (white circle, unmethylated CpG; black circle, methylated CpG; comma, indeterminate read).

Derivation of Therapeutic Cell Populations from CTX-iPSCs

It can be shown using RT-qPCR that differentiation along the three germline lineages (endoderm, mesoderm, ectoderm) is achieved. Differentiation of CTX-iPSCs to therapeutically-relevant cell types can also be confirmed. This has been demonstrated for adult stem cell types (mesenchymal stem cells). Other cell types can be generated by appropriate culture conditions, as will be apparent to the skilled person. In particular, cells of the immune system such as T lymphocytes, NK cells and dendritic cells can be differentiated by the methods disclosed in Themeli et al. (2013) Nature Biotechnology (31), 928-933.

FIG. 5 shows the production of a therapeutic cell population derived from CTX-iPSCs. (A) CTX-iPSCs on Laminin-521 in mTeSR1 medium. (B) Plastic-adherent candidate mesenchymal stem cells (MSCs) derived from cells in (A) in MSC medium (a-MEM, 10% FCS, 25 mM HEPES). (C) Flow cytometry of the CTX-iPSC-MSCs shows they express the MSC markers CD73, CD90 and CD105, but not CD14, CD20, CD34 or CD45, in accordance with ISCT criteria (blue, staining; red, isotype controls).

### CONCLUSION

Despite in vitro immortalisation and long term culture, it has surprisingly been shown that the neural stem cell line CTX0E03 can be reprogrammed by exogenous transcription factors.

CTX-iPSCs are apparently indistinguishable from conventional iPSCs generated from low passage primary cells, as defined by cellular morphology, expression of cell surface, transcription factor and enzymatic markers, and pluripotency.

The c-myc-ER^{TAM} locus in CTX-iPSCs remains active in at least some lines.

Clinically-relevant cell types (e.g. MSCs, immune cells such as T cells, NK cells and dendritic cells) may be generated from CTX-iPSCs

Induction of cell cycling via the 4-OHT/c-myc-ERTAM system in CTX-iPSC-MSCs could permit their scalable production for allogeneic therapy.

The CTX-iPSCs therefore represent a very useful clinical resource. They may be differentiated along a desired lineage to generate a target population such as a tissue progenitor cell type or adult stem cell population, and then provision of 4-OHT to promote continuous growth and prevent cell cycle exit and associated further differentiation could allow the routine and scalable production of previously-unattainable clinically-relevant subpopulations without repeated cell isolation from primary material.

Cloning or purification steps can be used to generate pure populations of the desired therapeutic types from more- or less-heterogeneous differentiation cultures for large-scale production of off-the-shelf treatments for conditions for which CTX itself is unsuitable, obviating the drawbacks seen on the art with incomplete efficiency of differentiation protocols. This applies to both the cells themselves or exosomal fractions produced by different cell types with alternative repertoires of payload molecules to those produced by CTX cells themselves.

Furthermore, as these CTX-iPSC-derivative sublines are derived from a cell line which has already passed clinical phase safety trials (CTX), their entry into clinical trials for efficacy in new indications is likely to be accelerated.

### Example 2: Characterisation of the Reprogrammed CTX-iPSCs

Reprogramming-induced modulation of expression of significant genes is shown, confirming that the CTX cells evidence have been properly reprogrammed.

The results are provided in FIG. 6. Each panel is a “tSNE” plot of single cell transcriptome data created from CTX. The key in the top left indicates that the green “cloud” is CTX, CTX-iPSCs are blue and CTX-iPSCs that have been subjected to a cortical differentiation protocol and then their transcriptome has been analysed when they are closest as possible to CTX itself are in red. Each cloud consists of dots representing a single cell. Grey: no expression, orange: moderate expression; red: high expression. The plots show that the pluripotency genes inactive in CTX have been activated in the reprogrammed cells: POU5F1, NANOG, UTF1, TET1, DPP4, TDGF1, ZSCAN10 and GAL. Importantly, of these genes only POU5F1 was provided exogeneously during reprogramming. Conversely, several neural genes expressed by CTX are downregulated upon reprogramming to pluripotency (NOGGIN, ADAM12, OCIAD2, NTRK3, PAX6. Finally, GLI3 (and to a great extent PAX6) are upregulated upon cortical differentiation of the pluripotent cells.

### Germ Lineage Differentiation and Staining Thereof of Induced Pluripotent Stem Cells (FIG. 7 and FIG. 9)

### Methods

1. CTX-iPSCs or STR0C05-iPSCs as appropriate were plated on human laminin-521-coated 8 well chamber slides. They were then treated with appropriate differentiation media (StemCell Technologies, cat. no. 05230) for 5-7 days as appropriate before fixation in 4% formaldehyde in phosphate buffered saline (PBS) and storage at 4° C. until immunostaining.
2. Wells were immunostained as follows:
   1. Blocked with normal goat serum (NGS) by incubation in 10% NGS/PBS for 30 minutes at room temperature.
   2. Wells were incubated with primary antibodies: Mouse anti-x and rabbit anti-y diluted as appropriate (see table below) in 0.1% PBST (0.1% Triton-X-100/PBS) for 2-4 hours at room temperature or overnight at 4° C.
   3. Wells were washed 3 times with PBS for 10 minutes, or kept overnight at 4° C. in PBS.
   4. Wells were incubated with secondary antibodies: Goat anti-mouse IgG-Alexafluor-488 (diluted 1:300) and/or Goat anti-rabbit IgG Alexafluor-568 (diluted 1:2000) in PBS for 2 hours at room temperature.
   5. Wells were washed 3 times with PBS at room temperature.
   6. Wells were stained with Hoechst 33342, diluted 1:10,000 in PBS, for 5 minutes.
   7. Wells were washed 3 times with PBS, for 5 minutes.
   8. The wells were removed from the slides, 2 drops of Vectashield was added and a glass cover slip placed on top, followed by examination by fluorescent microscopy.
3. The antibodies employed are shown in the table below.

| Lineage | Marker | Co. | Cat No. | Species | Isotype | Clone | Dilution |
|---|---|---|---|---|---|---|---|
| Endoderm | SOX17 | Abcam | ab84990 | Mouse | IgG1 | OTI3B10 | 1:100 |
| | FOXA2 | Abcam | ab108422 | Rabbit | IgG | EPR4466 | 1:500 |
| Mesoderm | BRACHYURY | Insightbio | sc-374321- | Mouse | IgG2b | A-4 | 1:150 |
| | | | AF488 | | | | |
| | CXCR4 | Abcam | ab124824 | Rabbit | IgG | UMB2 | 1:500 |
| Ectoderm | PAX6 | Abcam | ab5790 | Rabbit | IgG | Polyclonal | 1:50 |
| | NESTIN | Abcam | ab22035 | Mouse | IgG1 | 10C2 | 1:100 |

### Results:

Additional confirmation of the pluripotency of the CTX-iPSCs is provided by evidence of differentiation to endoderm, mesoderm and ectoderm, shown by coexpression of protein markers (mostly transcription factors) identifying the three primary germ layers. These data, in FIG. 7, are a complement to the RT-qPCR data previously shown.

### Example 3: Reprogramming of Fetal Striatal Cells

Another conditionally immortalised adult stem cell type was successfully reprogrammed. This line is STR0C05, derived from fetal striatal cells.

### Methods—Reprogramming of STR0C05 Cells to Pluripotency

1. An optimal range of transfection conditions specific for STR0C05 cells was identified, using the Neon electroporation instrument offered by Thermofisher.com. The frequency of live and green cells obtained was evaluated when a GFP expression plasmid was transfected into the cells using a range of different parameters such as voltage, pulse duration, etc., as suggested by the instrument manufacturer, to identify suitable transfection conditions for this cell line.
2. STR0C05 cells were then electroporated with the plasmids of the Epi5 reprogramming kit (Thermofisher cat. no. A15960; contains the reprogramming plasmids pCE-hOCT3/4, pCE-hSK, pCE-hUL and pCEmP53DD, expressing the transcription factors POU5F1, SOX2, KLF4, L-MYC, LIN28 and an dominant negative inhibitor of p53) using the conditions identified in (1), and plated onto human laminin-521. Wells were monitored daily with an Incucyte Zoom automated phase contrast microscope running inside the incubator.
3. After one week, the cells were either replated or remained in the same well, and medium was changed to mTeSR1 (StemCell Technologies cat. no. 85850).
4. Wells were monitored until pluripotent phenotypic colonies arose.
5. Once large enough, individual colonies were picked with a pipette tip into a well of a 24 well plate, also coated with hLn-521, and expanded until freezing or analysis.
6. As with previous work, alkaline phophatase staining was performed with the Stemgent alkaline phosphatase staining kit (cat. no. 00-0055) and flow cytometry for pluripotent stem cell markers such as SSEA1 and SSEA4 was performed with the Becton Dickinson Stemflow antibody kit (cat. no. 560477) supplemented with FITC-conjugated mouse anti-human TRA-1-60 antibody (BD cat. no. 560380), both according to the manufacturer's instructions. Flow cytometry samples were analysed on a Miltenyi MACSQuant 10 flow cytometer.

### Results

The results are shown in FIG. 8, wherein:
Panel A shows a colony of reprogrammed STR0C05 cells 24 days post-transfection with reprogramming factors;
Panel B shows alkaline phosphatase (red)-stained STR0C05 cells at an early stage of reprogramming, showing some express the pluripotency marker alkaline phosphatase;
Panel C shows the established STR0C05-iPSC line;
Panel D shows that AP-positive colonies appear at different frequencies in wells subjected to different transfection conditions; well 1 with no colonies was transfected with a GFP non-reprogramming plasmid as a control and had no reprogrammed cells, wells 4 and 6 had few surviving cells;
Panel E shows that established STR0C05-iPSC lines is alkaline phosphatase positive; and
Panel F shows that it is also positive for the pluripotency marker SSEA4 but negative for the early differentiation marker SSEA1.

The pluripotency of the STR0C05-iPSCs is also confirmed, using the Germ lineage Differentiation method described in Example 2 above and with the results shown in FIG. 9. Differentiation is demonstrated to endoderm, mesoderm and ectoderm, shown by coexpression of protein markers (mostly transcription factors) identifying the three primary germ layers, as FIG. 7 for CTX.

### Example 4: Adult Stem Cells Derived from the Reprogrammed iPSCs are Multipotent

The multipotency of adult stem cells derived from CTX-iPSCs was confirmed. Previously we have shown example flow cytometry profiles showing appropriate marker expression and the ability to adhere to plastic for candidate CTX-iPSC-MSCs (mesenchymal stem cells). This experiment confirms the ability of the CTX-iPSC-MSCs to differentiate into several different cell types.

### Methods—Differentiation of CTX-iPSC-MSCs to Confirm Multipotency

1. For assessment of fat and bone cell formation, CTX-iPSC-MSCs were plated in 6 well tissue culture-treated plates and incubated for up to 28 days with commercially-available media promoting adipogenesis and osteogenesis (adipogenesis: StemCell Technologies cat. no. 05412, osteogenesis: StemCell Technologies cat. no. 05465 or R&D systems cat. nos. CCMN007 and CCM008), prior to fixation and staining. For assessment of cartilage formation, CTX-iPSC-MSCs were pelleted as clumps in the bottom of a 15 ml tube and cultured with chondrogenic medium (StemCell Technologies cat. no. 05455) followed by formaldehyde fixation, paraffin embedding and sectioning using standard methods.
2. Alcian Blue Staining (chondrogenesis): Sections on slides were hydrated to distilled water, treated with 3% acetic acid for 3 minutes and then stained with 1% Alcian blue in 3% acetic acid, pH 2.5, for 30 minutes. The slides were then washed in running water for 5 minutes, rinsed in distilled water and counterstained for 5 minutes with 0.1% nuclear fast red in 5% aluminium sulphate solution prior to imaging.
3. Oil Red 0 Staining (adipogenesis): Cells in the 6 well plate were washed with PBS, fixed with 10% formaldehyde for 10 minutes at room temperature and washed twice with PBS. They were stained for 15 minutes in 0.3% oil red 0 in 60% isopropanol/40% water and washed with double distilled water prior to imaging.
4. Alizarin Red S Staining (osteogenesis): Cells in the 6 well plate were washed with PBS, fixed with 10% formaldehyde for 10 minutes at room temperature and washed twice with PBS. They were stained for 15 minutes at room temperature with 2% alizarin red S solution, pH 4.2, washed with water and imaged.

### Results

FIG. 10 shows the capacity of the iPSC-derived MSCs to differentiate into cartilage (shown by alcian blue staining of sialoglycans), fat (shown by staining of intracellular lipid droplets with oil red O) and bone (shown by alizarin red staining of deposited calcium).

Flow cytometric profiles were then obtained for CTX-iPSC-MSCs cultured to high passage (20 passages) in the presence or absence of 4-OHT. The results are shown in FIG. 11. The tested line is one for which the previously generated bisulphite data indicated had a demethylated C-MYC-ER^{TAM} promoter, in turn suggesting the promoter should still be active in these cells. Interestingly, this line appears to better maintain its marker profile when 4-OHT is inducing cell cycling: CD90 and CD105 expression are more uniform and higher, and the negative markers CD14, 20, 34 and 45 are more tightly “off”. (This line always shows lower CD73 expression, possibly an antibody artefact.) In the second panel, the 4-OHT-treated cells appear to be more efficient at generating bone upon differentiation, suggesting that 4-OHT-mediated forcing of cell cycling ameliorates exit from the cycle and loss of potency.

### Example 5: Further Characterisation of the Reprogrammed CTX-iPSC-MSCs

CTX-iPSC-MSC lines were cultured in the absence or presence of 4-OHT. The results from experiments with two different CTX-iPSC-MSC cell cultures in FIGS. 12 and 13 show improved and more consistent growth long-term with 4-OHT/C-MYC-ERTAM present and active.

This Example shows that conditionally-immortalised iPSC-ASCs may be propagated more reliably and for longer.

### SELECTED REFERENCES

Banerjee, S., Williamson, D., Habib, N., Gordon, M., Chataway, J. (2011) Age and Ageing 40:7-Chung et al., Cell Stem Cell, 2, 113-117, 2008
Einstein, O., Ben-Hur, T. (2008) Arch Neurol 65:452-456
Gennaro (2000) Remington: The Science and Practice of Pharmacy. 20th edition, ISBN: 0683306472
Hassani Z, O'Reilly J, Pearse Y, Stroemer P, Tang E, Sinden J, Price J, Thuret S. “Human neural progenitor cell engraftment increases neurogenesis and microglial recruitment in the brain of rats with stroke.” PLoS One. 2012; 7(11):e50444. doi: 10.1371/journal.pone.0050444. Epub 2012 Nov. 21.
Hodges et al. Cell Transplant. 2007; 16(2):101-15
Horie, N., Pereira, N. P., Niizuma, K. Sun, G. et al. (2011) Stem Cells 29:274-285.
Kornblum, Stroke 2007, 38:810-816
Littlewood, T. D., Hancock, D. C., Danielian, P. S. et al. (1995) Nucleic Acid Research 23:1686-1690.
Miljan, E. A. & Sinden, J. D. (2009) Current Opinion in Molecular Therapeutics 4:394-403
Miljan E A, Hines S J, Pande P, Corteling R L, Hicks C, Zbarsky V, Umachandran, M, Sowinski P,
Richardson S, Tang E, Wieruszew M, Patel S, Stroemer P, Sinden J D. Implantation of c-mycER TAM immortalized human mesencephalic-derived clonal cell lines ameliorates behavior dysfunction in a rat model of Parkinson's disease. Stem Cells Dev. 2009 March; 18(2):307-19
Pollock et al, Exp Neurol. 2006 May; 199(1):143-55.
Smith, E. J., Stroemer, R. P., Gorenkova, N., Nakajima, M. et al. (2012) Stem Cells 30:785-796.
Stevenato, L., Corteling, R., Stroemer, P., Hope, A. et al. (2009) BMC Neuroscience 10:86
Stroemer, P., Patel, S., Hope, A., Oliveira, C., Pollock, K., Sinden, J. (2009) Neurorehabil Neural Repair 23: 895-909.
Their et al, “Direct Conversion of Fibroblasts into Stably Expandable Neural Stem Cells”. Cell Stem Cell. 2012 Mar 20.
Themeli et al. “Generation of tumour-targeted human T lymphocytes from induced pluripotent stem cells for cancer therapy” Nature Biotechnology 2013 (31), 928-933.

1. An induced pluripotent stem cell comprising a controllable transgene for conditional immortalisation.

2. A pluripotent stem cell obtainable or obtained from a conditionally-immortalised cell or a conditionally immortalized stem cell.

3. The pluripotent stem cell according to claim 1, which is obtainable or obtained by reprogramming a conditionally-immortalized stem cell with one or more transcription factors.

4. The pluripotent stem cell according to claim 1, comprising the C-MYC-ER fusion protein.

5. The pluripotent stem cell according to claim 1, comprising the c-mycER transgene, optionally in its genome.

6. The pluripotent stem cell according to claim 1, which is obtainable or obtained from a conditionally-immortalized neural stem cell.

7. The pluripotent stem cell according to claim 1, which is obtainable or obtained from a conditionally-immortalized stem cell line.

8. The cell according to claim 7, wherein the stem cell line is CTX0E03 having ECACC Accession No. 04091601 or STR0005 having ECACC Accession No. 04110301.

9. A cell that is derived from the pluripotent cell of claim 1.

10. The cell according to claim 9, wherein the derived cell is a stem cell that expresses one or more markers of differentiation.

11. The cell according to claim 9, wherein the derived cell is of the endoderm, mesoderm or ectoderm lineage.

12. The cell according to claim 9, wherein the derived cell is:
a mesenchymal stem cell, a neural stem cell, or a haematopoietic stem cell;
a somatic (adult) stem cell;
a multipotent cell, an oligopotent cell or a unipotent cell;
a terminally-differentiated cell;
an immune cell, optionally selected from the list consisting of a T cell, an NK cell, a B cell, or a dendritic cell;
a cartilage cell;
a fat cell; or
a bone cell.

13. A method of producing a pluripotent stem cell, the method comprising the step of reprogramming a conditionally-immortalized stem cell.

14. The method according to claim 13, wherein the reprogramming comprises introducing one or more of the transcription factors OCT4, L-MYC, KLF4 and SOX2, and optionally the RNA-binding LIN28, into the conditionally-immortalised stem cell.

15. The method according to claim 14, wherein:
the introduced transcription factor comprises or consists of OCT4;
the introduced transcription factors comprise or consist of OCT4 and SOX2;
the introduced transcription factors comprise or consist of OCT, KLF4 and SOX2;
the introduced transcription factors comprise or consist of OCT4, KLF4, SOX2 and MYC; or
MYC activity is provided to promote the reprogramming process by the provision of 4-OHT in the medium to activate a c-myc-ER^{TAM} transgene in the stem cell to be reprogrammed.

16. The method according to claim 14, wherein the transcription factors and optional LIN28 are introduced into the conditionally-immortalized stem cell using one or more episomal plasmids or one or more viral vectors optionally selected from lentivirus, retrovirus or Sendai-virus, or by mRNA transfection.

17. The method according to claim 13, further comprising the step of differentiating the pluripotent stem cell down the endoderm, mesoderm or ectoderm lineage.

18. The method according to claim 17, wherein the endoderm, mesoderm or ectoderm lineage differs from the lineage of the conditionally-immortalized stem cell that was reprogrammed.

19. The method according to claim 17, wherein the pluripotent stem cell is differentiated into:
a mesenchymal stem cell, a neural stem cell, or a haematopoietic stem cell;
a somatic (adult) stem cell;
a multipotent cell, an oligopotent cell or a unipotent cell;
a terminally-differentiated cell;
an immune cell, optionally selected from the list consisting of a T cell, an NK cell, a B cell or a dendritic cell;
a cartilage cell;
a fat cell; or
a bone cell.

20. The method according to claim 17, further comprising the step of reactivating the conditionally-immortalized phenotype of the cell that results from the method.

21. The method according to claim 13, wherein the conditionally-immortalized stem cell that is reprogrammed is as defined in claim 3.

22. The method according to claim 13, further comprising one or more steps selected from the list consisting of:
culturing the cells that result from the method;
passaging the cells that result from the method;
harvesting or collecting the cells that result from the method;
packaging the cells that result from the method into one or more containers;
and
formulating the cells that result from the method with one or more excipients, stabilizers or preservatives.

23. A pluripotent stem cell obtained or obtainable by the method of claim 13.

24. A cell obtained or obtainable by the method of claim 17.

25. A microparticle produced by the cell of claim 1.

26. A microparticle according to claim 25, which is an exosome.

27. A pharmaceutical composition comprising a cell according to claim 1 or a microparticle according to claim 25, and one or more pharmaceutically acceptable excipients.

28. (canceled)
